(19) United States
(12) Patent Application Publication
(10) Pub. No.: US 2022/0193196 A1
(43) Pub. Date: Jun. 23, 2022

(54) Methods for Modulating T Cell Activation

(71) Applicant: THE WALTER AND ELIZA HALL INSTITUTE OF MEDICAL RESEARCH, Parkville (AU)

(72) Inventors: Edwin Hawkins, Parkville (AU); Najoua Lalaoui, Parkville (AU); Amanda Elizabeth Light, Parkville (AU); Mark McKenzie, Parkville (AU); John Silke, Parkville (AU)

(21) Appl. No.: 17/611,839

(22) PCT Filed: May 27, 2020
(86) PCT No.: PCT/AU2020/050528

§ 371 Date: Nov. 16, 2021

(30) Foreign Application Priority Data
May 28, 2019 (AU) 2019901818

(51) Int. Cl. A61K 38/19; C07K 14/705; A61K 38/05

(52) U.S. Cl. CPC A61K 38/191; A61K 38/05; C07K 14/70578

(57) ABSTRACT

The present invention relates to methods, compositions, and kits for increasing the activation of effector T cells in a subject or inhibiting the activation of effector T cells in a subject by increasing or decreasing TNFR2 (CD 120b) signaling respectively. The present invention also relates to methods, compositions, and kits for treating diseases such as cancer, infections, and autoimmune diseases.

### FIELD OF THE INVENTION

The present invention relates to methods, compositions, and kits for increasing or inhibiting activation of effector T cells in a subject. The present invention also relates to methods, compositions, and kits for treating diseases such as cancer, infections, and autoimmune diseases.

### BACKGROUND OF THE INVENTION

Tumour necrosis factor alpha (TNFα) elicits its biological activities by stimulation of two receptors, TNFR1 and TNFR2, both belonging to the TNF receptor superfamily. Effector T cells express both TNFR1 and TNFR2 on their cell membranes and play a central role in cell-mediated immunity through induction of apoptosis of diseased cells and the secretion of cytokines which activate other immune cells against disease. Activation of effector T cells occurs through simultaneous engagement of the T cell receptor (TCR) with its cognate peptide presented by major histocompatibility complex (MHCII) on antigen presenting cells (APCs) and co-stimulation through co-stimulatory receptors such as CD28. Activation of effector T cells is required for effective immune defence against cancer and infections, whereas over-activation of effector T cells is associated with a number of auto-immune and inflammatory diseases.

There have been a number of conflicting reports in the literature regarding the roles of TNFR1 and TNFR2 in T cell activation. For example, there have been studies to suggest that TNFR2 agonists can be used to suppress effector T cell activation in graft vs host disease (GVHD; see Chopra et al., 2016). In addition, Vanamee and Faustman (2017) suggest that blocking TNFR2 signaling can be used to treat cancer by inhibiting regulatory T cells (T_{reg}s).

There is therefore a need for new methods for selectively increasing and inhibiting effector T cell activation. Such methods would be useful for treating diseases such as cancer, infections, inflammatory diseases, and autoimmune diseases, for example.

### SUMMARY OF THE INVENTION

The present inventors have proceeded against prior teachings that TNFR2 signaling reduces the effector T cell response by activating T_{reg} cells. Specifically, the present inventors have identified that effector T cell responses can be activated or deactivated by increasing or decreasing TNFR2 (CD120b) signaling respectively. This provides the rationale for generating materials to specifically activate TNFR2 signaling to enhance activation of T cells to treat diseases which would benefit from an enhanced T cell response, for example.

Accordingly, in an aspect, the present invention provides a method of increasing activation of effector T cells in a subject, the method comprising administering a TNFR2-specific agonist to the subject.

In a related aspect, the present invention provides use of a TNFR2-specific agonist in the manufacture of a medicament for increasing activation of effector T cells in a subject.

In another related aspect, the present invention provides a TNFR2-specific agonist for use in increasing activation of effector T cells in a subject.

In some embodiments, the subject has a disease which would benefit from an increase in activation of effector T cells and the TNFR2-specific agonist is administered to treat the disease. Thus, advantageously, the methods of the invention can be used to enhance the subject's own immune response against the disease. In some embodiments, the disease is a cancer, infection, or immunodeficiency.

Any cancer for which an enhanced effector T cell response would be beneficial can be treated using the methods of the invention. In some embodiments, the cancer is a solid tumour, such as a sarcoma, carcinoma, or lymphoma, for example. In some embodiments, the cancer is a blood cancer, such as a leukemia, for example. In some embodiments, the cancer is lung cancer, breast cancer, colorectal cancer, prostate cancer, skin cancer (non-melanoma), melanoma, stomach cancer, pancreatic cancer, liver cancer, brain cancer, glioblastoma, neuroblastoma, blood cancer, acute myeloid leukaemia, acute lymphoblastic leukaemia, chronic lymphocytic leukemia, chronic myelogenous leukemia, a chronic myeloproliferative neoplasm, parathyroid cancer, renal cancer, retinoblastoma, rhabdomyosarcoma, salivary gland cancer, sarcoma, TNF-secreting T cell lymphoma, throat cancer, thymoma, thymic carcinoma, wilms tumour, hodgkin lymphoma, non-hodgkin lymphoma, merkel cell carcinoma, esophageal cancer, bladder cancer, bile duct cancer, bone cancer, ewing sarcoma, osteosarcoma, malignant fibrous histiocytoma, multiple myeloma, or ovarian cancer.

In some embodiments, the infection is an acute or a chronic infection. In some embodiments, the infection is a tuberculosis, influenza, mycobacterium uclercans, hepatitis, herpes simplex virus, ebola virus, human immunodeficiency virus, encephalitis, burkholderia pseudomallei, legionellosis, leishmaniasis, listeriosis, malaria, measles, meningococcal meningitis, pneumonia, salmonella, rubella, rabies, tetanus, typhoid, west nile virus, zika virus, anthrax, dengue fever, brucellosis, or campylobacter infection.

In some embodiments the TNFR2-specific agonist is a polypeptide. In some embodiments the TNFR2-specific agonist is a small molecule. In some embodiments the TNFR2-specific agonist is a polynucleotide.

In some embodiments, the TNFR2-specific agonist is or comprises a TNFR2-specific TNF mutein. Wild type TNFα preferentially activates TNFR1 signaling, rather than TNFR2 signaling and is therefore not a suitable TNFR2-specific agonist as described herein. However, it is known in the art that TNFα can be mutated to alter its specificity for TNFR1 or TNFR2. Thus, the methods of the invention encompass the use of mutated forms of TNFα, i.e. “TNF muteins”, which have specificity for TNFR2, rather than TNFR1.

In some embodiments, the TNFR2-specific TNF mutein is mSTAR2, hSTAR2, sc-mTNF_{R2}, EHD2-sc-hTNF_{R2}, p53-sc-mTNF_{R2}, GCN4-sc-mTNF_{R2}, TNC-scTNF_{R2}, or TNF (D143N/A145R). In some embodiments, the TNFR2-specific mutein is MHD2-scTNFR2, EHD2-scTNFR2-L16aa, EHD2-scTNFR2-L28aa, sc-hTNF_{R2}, or EHD2-sc-mTNF_{R2}.

In some embodiments, the TNFR2-specific agonist is a polypeptide comprising a sequence which is at least 70% or at least 80% or at least 90% or at least 95% identical to a sequence set forth in any one of SEQ ID NOs: 2-14, or a biologically active fragment thereof.

In some embodiments, the TNFR2-specific agonist is a polypeptide comprising a sequence which is at least 70% or at least 80% or at least 90% or at least 95% identical to SEQ ID NO:2 or SEQ ID NO:3 or a biologically active fragment thereof. In some embodiments, the TNFR2-specific agonist is hSTAR2 (human STAR2) or mSTAR2 (murine STAR2).

In some embodiments, the TNFR2-specific agonist is a polypeptide comprising a sequence which is at least 70% or at least 80% or at least 90% or at least 95% identical to SEQ ID NO:4 or a biologically active fragment thereof. Thus, in some embodiments, the TNFR2-specific agonist is EHD2-sc-hTNF_{R2}.

In some embodiments, the TNFR2-specific agonist is a polypeptide comprising a sequence which is at least 70% or at least 80% or at least 90% or at least 95% identical to SEQ ID NO:5 or a biologically active fragment thereof. Thus, in some embodiments, the TNFR2-specific agonist is TNC-scTNF_{R2}.

In some embodiments, the TNFR2-specific agonist is a polypeptide that comprises an antigen-binding domain which binds to TNFR2. In some embodiments, the polypeptide is an antibody. Thus, in some embodiments, the TNFR2-specific agonist is an agonistic antibody. Such antibodies, or fragments thereof, may specifically bind to TNFR2 and activate TNFR2-mediated signaling. In some embodiments, the TNFR2-specific agonist antibody preferentially activates TNFR2 signaling relative to TNFR1 signaling. In some embodiments, the TNFR2-specific agonist antibody binds with higher affinity to TNFR2 relative to TNFR1.

In some embodiments, the TNFR2-specific agonist is administered to the subject in combination with another compound or cell. In some embodiments, the other compound or cell is one which is capable of increasing activation of effector T cells. In some embodiments, the other compound or cell is a therapy which is enhanced by the TNFR2-specific agonist. In one embodiment, the other compound is a polypeptide. In one embodiment, the other compound is a small molecule. In one embodiment, the other compound is a polynucleotide.

In some embodiments, the other compound or cell is an immunotherapy. In some embodiments, the immunotherapy is an antibody. In some embodiments, the immunotherapy is a cell-based therapy.

In some embodiments, the immunotherapy is a T cell-based immunotherapy. In some embodiments, the T cell-based immunotherapy is a CAR-T cell therapy. Thus, in some embodiments, the TNFR2-specific agonist enhances the CAR-T cell therapy by increasing CAR-T cell activation.

In some embodiments, the other compound or cell is a vaccine. In some embodiments, the TNFR2-specific agonist is administered as a vaccine adjuvant. In some embodiments, the other compound or cell is a vaccine and the TNFR2-specific agonist is administered as a vaccine adjuvant. Thus, the TNFR2-specific agonist can be used to enhance the immune response elicited by the vaccine. In some embodiments, the vaccine is a T cell vaccine.

In some embodiments, the other compound is a compound which synergistically increases effector T cell activation when administered in combination with the TNFR2-specific agonist. For example, the present inventors have surprisingly found that SMAC mimetics synergistically increase effector T cell activation when administered in combination with the TNFR2-specific agonist. Thus, in some embodiments, the other compound is a SMAC mimetic.

In some embodiments, the SMAC mimetic is birinapant, Debio 1143, CUDC-427, LCL161, AEG40826, ASTX-660, LBW-242, AZD5582, AEG40730, APG-1387, CompA, GDC-0145, GDC-0152, CS3, BV6, MV1, SM-164, AT406, ML101, or embelin. In some embodiments, the SMAC mimetic is birinapant.

In some embodiments, the other compound is a T cell receptor (TCR) agonist. In some embodiments, the TCR agonist is anti-CD3 antibody, phytohaemaglutinin (PHA), phorbol myristate acetate (PMA), or ionomycin. In some embodiments, the T cell receptor agonist is a Tribody. In some embodiments, the tribody is Tb535.

In some embodiments, the other compound is a checkpoint inhibitor. Such checkpoint inhibitors are useful when administered in combination with the TNFR2-specific agonist for the treatment of cancer. For example, in some embodiments, the TNFR2-specific agonist increases effector T cell activation and the checkpoint inhibitor blocks T cell inhibition by inhibitory checkpoint molecules expressed by a cancer cell (e.g., a tumour cell).

In some embodiments, the checkpoint inhibitor is a CTLA-4 antagonist, PD-1 antagonist, or PD-L1 antagonist. In some embodiments, the checkpoint inhibitor is pembrolizumab, ipilimumab, nivolumab, or atezolizumab.

The present inventors have also found that activation of effector T cells can be reduced by inhibiting TNFR2 signaling. Thus, in another aspect, the present invention provides a method of inhibiting activation of T cells in a subject, the method comprising administering a TNFR2-specific antagonist to the subject.

In a related aspect, the present invention provides use of a TNFR2-specific antagonist in the manufacture of a medicament for inhibiting activation of effector T cells in a subject.

In another related aspect, the present invention provides a TNFR2-specific antagonist for use in inhibiting activation of effector T cells in a subject.

In some embodiments, the subject has a disease mediated by over-activation of effector T cells, and the TNFR2-specific antagonist is administered to the subject to treat the disease. In some embodiments, the disease is an autoimmune disease, inflammatory disease, or a non-TNF secreting T cell lymphoma.

In some embodiments, the disease is graft versus host disease, inflammatory bowel disease, ulcerative colitis, lupus, polyarthritis, rheumatoid arthritis, reactive arthritis, osteomyelitis, toxic shock syndrome, psoriasis, Hidradenitis Suppurativa, ankylosing spondylitis, asthma, type 1 diabetes, type 2 diabetes, cardiovascular disease, or vasculitis.

In some embodiments, the TNFR2-specific antagonist is a polypeptide. In some embodiments, the TNFR2-specific antagonist is a small molecule. In some embodiments, the TNFR2-specific antagonist is a polynucleotide.

In some embodiments, the TNFR2-specific antagonist is a competitive inhibitor of TNFR2 signaling. For example, in some embodiments, the TNFR2-specific antagonist competitively inhibits TNFα binding to TNFR2. In other embodiments, the TNFR2-specific antagonist is a non-competitive inhibitor of TNFR2 signaling.

Wild type TNFα is capable of binding to TNFR2 and inducing TNFR2 signaling. However, it is known in the art that TNFα can be mutated to alter its binding specificity and its ability to activate TNFR2 signaling. Thus, the methods of the invention encompass the use of mutated forms of TNFα, i.e. “TNF muteins”, which have binding specificity for TNFR2 but which are capable of inhibiting TNFR2 signaling, rather than activating TNFR2 signaling like wild type TNFα. Accordingly, in some embodiments, the TNFR2-specific antagonist is a TNFα mutein.

In some embodiments, the TNFR2-specific antagonist is a polypeptide that comprises an antigen-binding domain of an antibody. In some embodiments, the antibody is an anti-TNFR2 antibody. In some embodiments, the antigen binding domain comprises the CDRs of M861 or TR75-54.7. In some embodiments, the antibody comprises a heavy chain variable region (V_{H}) and/or light chain variable region (V_{L}) which has an amino acid sequence which is at least 60% or at least 70% or at least 80% or at least 90% or at least 95% or at least 98% identical to the V_{H} and/or V_{L} of M861 or TR75-54.7. In some embodiments, the antibody is M861 or TR75-54.7.

In one embodiment, the TNFR2-specific antagonist reduces the expression of TNFR2. For instance, the TNFR2-specific antagonist may reduce the expression of TNFR2 by reducing TNFR2 mRNA levels by, for example, RNA interference. Thus, in one embodiment, the TNFR2-specific antagonist is an siRNA.

In some embodiments, the TNFR2-specific antagonist is administered in combination with another compound or cell. In some embodiments, the other compound is one which is capable of inhibiting activation of effector T cells. In one embodiment, the other compound is a polypeptide. In one embodiment, the other compound is a small molecule. In one embodiment, the other compound is a polynucleotide.

In some embodiments, the other compound is a TNFα antagonist. For example, in some embodiments, the TNFα antagonist is a polypeptide which comprises an antigen-binding domain of an antibody that binds to TNFα. In some embodiments, the TNFα antagonist is adalimumab, etanercept, golimumab, certolizumab, or infliximab.

In some embodiments, the other compound is a CD52 antagonist, CD20 antagonist, or IL-17A antagonist. In some embodiments, the CD52 antagonist is alemtuzumab. In some embodiments, the CD20 antagonist is rituximab. In some embodiments, the IL-17A antagonist is ixekizumab.

In some embodiments, the subject is a human. In some embodiments, the subject is a non-human animal. In some embodiments, the subject is a mammal.

The present invention can also be used for in vitro methods of increasing or inhibiting the activation and proliferation of effector T cells. Thus, in another aspect, the present invention provides an in vitro method of expanding a population of effector T cells, the method comprising contacting the effector T cells with a TNFR2-specific agonist.

In some embodiments, the effector T cells are harvested from a subject. Thus, the present invention can be used as part of a T cell therapy in which T cells are harvested from a subject, subsequently expanded in vitro (i.e., ex vivo) using the methods of the invention, and then re-administered to the subject.

Thus, in another aspect, the present invention provides a method comprising harvesting effector T cells from a subject, expanding the harvested effector T cells by contacting them with a TNFR2-specific agonist, and subsequently re-administering the expanded effector T cells to the subject.

In some embodiments, the expanded effector T cells are CAR-T cells. For example, the T cells may be harvested from a subject, then engineered to express a chimeric antigen receptor (CAR), then expanded by contacting them with a TNFR2-specific agonist, and subsequently re-administered to the subject.

In another aspect, the present invention provides a composition comprising at least two agents for increasing activation of effector T cells, wherein one of the agents is a TNFR2-specific agonist.

In another aspect, the present invention provides a composition comprising at least two agents for treating cancer, infection or an immunodeficiency, wherein one of the agents is a TNFR2-specific agonist.

In another aspect, the present invention provides a composition comprising at least two agents for inhibiting activation of T cells in a subject, wherein one of the agents is a TNFR2-specific antagonist.

In another aspect, the present invention provides a composition comprising at least two agents for treating an autoimmune disease, inflammatory disease or non-TNF secreting T cell lymphoma, wherein one of the agents is a TNFR2-specific antagonist.

In another aspect, the present invention provides a kit comprising at least two agents for increasing activation of effector T cells, wherein one of the agents is a TNFR2-specific agonist.

In another aspect, the present invention provides a kit comprising at least two agents for treating cancer, infection or an immunodeficiency, wherein one of the agents is a TNFR2-specific agonist.

In another aspect, the present invention provides a kit comprising at least two agents for inhibiting activation of T cells in a subject, wherein one of the agents is a TNFR2-specific antagonist.

In another aspect, the present invention provides a kit comprising at least two agents for treating an autoimmune disease, inflammatory disease or non-TNF secreting T cell lymphoma, wherein one of the agents is a TNFR2-specific antagonist.

In some embodiments, the TNFR2-specific agonist

a) binds to TNFR2 with an affinity which is at least 1.2-fold, at least 1.5-fold, at least 2-fold, at least 5-fold, or at least 10-fold stronger than its affinity for binding to TNFR1, and/or

b) activates TNFR2 signaling at a level which is at least 1.2-fold, at least 1.5-fold, at least 2-fold, at least 5-fold, or at least 10-fold higher than it activates TNFR1 signaling.

In some embodiments, the TNFR2-specific agonist does not detectably bind TNFR1 and/or does not activate any detectable level of TNFR1 signaling.

In some embodiments, the TNFR2-specific antagonist

a) binds to TNFR2 with an affinity which is at least 1.2-fold, at least 1.5-fold, at least 2-fold, at least 5-fold, or at least 10-fold stronger than its affinity for binding to TNFR1, and/or

b) inhibits TNFR2 signaling at a level of inhibition which is at least at least 1.2-fold, 1.5-fold, at least 2-fold, at least 5-fold, or at least 10-fold higher than it inhibits TNFR1 signaling.

In some embodiments, the TNFR2-specific antagonist does not detectably bind TNFR1 and/or does not inhibit TNFR1 signaling by a detectable amount.

Any embodiment herein shall be taken to apply mutatis mutandis to any other embodiment unless specifically stated otherwise. For instance, as the skilled person would understand, examples of agonists and antagonists outlined above for the methods of the invention equally apply to the use, kits, and compositions of the invention.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### BRIEF DESCRIPTION OF THE ACCOMPANYING DRAWINGS

FIG. 1—TNF promotes CD4 T cell division and expansion: Naïve mouse CD4 T cells were activated via TCR with anti-CD3 (10 μg/ml) in the presence of titrated doses of soluble recombinant TNF. The response was measured in vitro over 4 days via total cell numbers. Data represent mean±S.E.M for triplicate samples. Representative cell division plots for data in FIG. 1 are shown in FIG. 2.

FIG. 2—TNF promotes CD4 T cell division and expansion: Naïve mouse CD4 T cells were activated via TCR with anti-CD3 (10 μg/ml) in the presence of titrated doses of soluble recombinant TNF. The response was measured in vitro over 4 days via dilution of cell tracking dyes (shown for 72 hours).

FIG. 3—TNF is a potent co-stimulation factor to promote CD8 T cell division and expansion: Naïve mouse CD8 T cells were activated via TCR with anti-CD3 (10 μg/ml) in the presence of titrated doses of soluble recombinant TNF. The response was measured in vitro over 4 days via total cell numbers. Data represent mean±S.E.M for triplicate samples. Representative cell division plots for data in FIG. 3 are shown in FIG. 4.

FIG. 4—TNF is a potent co-stimulation factor to promote CD8 T cell division and expansion: Naïve mouse CD8 T cells were activated via TCR with anti-CD3 (10 μg/ml) in the presence of titrated doses of soluble recombinant TNF. The response was measured in vitro over 4 days via dilution of cell tracking dyes (shown for 72 hours).

FIG. 5—TNF co-stimulates CD4 T cells through TNFR2 to promote cell division: Naïve mouse CD4 T cells were isolated from WT, Tnfr 1−/− and Tnfr2−/− mice activated via TCR with anti-CD3 (10 μg/ml) in the presence of recombinant TNF (5 ng/ml). The response was measured in vitro over 4 days via total cell numbers. Data represent mean±S.E.M for triplicate samples in A. Representative cell division plots for data in FIG. 5 are shown in FIGS. 6 to 9.

FIG. 6—TNF co-stimulates CD4 T cells through TNFR2 to promote cell division: Naïve mouse CD4 T cells were isolated from WT mice activated via TCR with anti-CD3 (10 μg/ml) in the presence of recombinant TNF (5 ng/ml). The response was measured in vitro over 4 days via dilution of cell tracking dyes (shown for 72 hours).

FIG. 7—TNF co-stimulates CD4 T cells through TNFR2 to promote cell division: Naïve mouse CD4 T cells were isolated from Tnfr1−/− mice activated via TCR with anti-CD3 (10 μg/ml) in the presence of recombinant TNF (5 ng/ml). The response was measured in vitro over 4 days via dilution of cell tracking dyes (shown for 72 hours).

FIG. 8—TNF co-stimulates CD4 T cells through TNFR2 to promote cell division: Naïve mouse CD4 T cells were isolated from and Tnfr2−/− mice activated via TCR with anti-CD3 (10 μg/ml) in the presence of recombinant TNF (5 ng/ml). The response was measured in vitro over 4 days via dilution of cell tracking dyes (shown for 72 hours).

FIG. 9—TNF co-stimulates CD4 T cells through TNFR2 to promote cell division: Naïve mouse CD4 T cells were isolated from WT and Tnfr2−/− mice activated via TCR with anti-CD3 (10 μg/ml) in the presence of recombinant TNF (5 ng/ml). The response was measured in vitro over 4 days via dilution of cell tracking dyes (shown for 72 hours).

FIG. 10—TNF co-stimulates CD8 T cells through TNFR2 to promote cell division: Naïve mouse CD8 T cells were isolated from WT, Tnfr1−/− and Tnfr2−/− mice and activated via TCR with anti-CD3 (10 μg/ml) in the presence of recombinant TNF (5 ng/ml). The response was measured in vitro over 4 days via total cell numbers. Data represent mean±S.E.M for triplicate samples. Representative cell division plots for data in FIG. 10 are shown in FIGS. 11 to 14.

FIG. 11—TNF co-stimulates CD8 T cells through TNFR2 to promote cell division: Naïve mouse CD8 T cells were isolated from WT mice and activated via TCR with anti-CD3 (10 μg/ml) in the presence of recombinant TNF (5 ng/ml). The response was measured in vitro over 4 days via dilution of cell tracking dyes (shown for 72 hours).

FIG. 12—TNF co-stimulates CD8 T cells through TNFR2 to promote cell division: Naïve mouse CD8 T cells were isolated from Tnfr1−/− mice and activated via TCR with anti-CD3 (10 μg/ml) in the presence of recombinant TNF (5 ng/ml). The response was measured in vitro over 4 days via dilution of cell tracking dyes (shown for 72 hours).

FIG. 13—TNF co-stimulates CD8 T cells through TNFR2 to promote cell division: Naïve mouse CD8 T cells were isolated from Tnfr2−/− mice and activated via TCR with anti-CD3 (10 μg/ml) in the presence of recombinant TNF (5 ng/ml). The response was measured in vitro over 4 days via dilution of cell tracking dyes (shown for 72 hours).

FIG. 14—TNF co-stimulates CD8 T cells through TNFR2 to promote cell division: Naïve mouse CD8 T cells were isolated from WT and Tnfr2−/− mice and activated via TCR with anti-CD3 (10 μg/ml) in the presence of recombinant TNF (5 ng/ml). The response was measured in vitro over 4 days via dilution of cell tracking dyes (shown for 72 hours).

FIG. 15—TNF co-stimulation can synergise with Smac-mimetics to promote T-cell division and expansion: Naïve mouse CD8 T cells were activated via TCR with anti-CD3 (10 μg/ml) in the presence of combinations of recombinant TNF (5 ng/ml) and SMAC mimetics (500 μM). The response was measured in vitro over 4 days via total cell numbers. Data represent mean±S.E.M for triplicate samples.

FIG. 16—The TNFR2-specific agonist mSTAR can mimic TNF mediated CD4 T cell activation: Mouse CD4 T cells were activated via TCR with anti-CD3 (10 μg/ml) in the presence and absence of TNF (5 ng/ml). The response was measured at 72 hours by dilution of cell division dyes. Representative cell division plots are shown in FIG. 19.

FIG. 17—The TNFR2-specific agonist mSTAR can mimic TNF mediated CD4 T cell activation: Mouse CD4 T cells were activated via TCR with anti-CD3 (10 μg/ml) in the presence and absence of mSTAR, a TNFR2-specific agonist (5 ng/ml). The response was measured at 72 hours by dilution of cell division dyes. Representative cell division plots are shown in FIG. 19.

FIG. 18—The TNFR2-specific agonist mSTAR can mimic TNF mediated CD4 T cell activation: Mouse CD4 T cells were activated via TCR with anti-CD3 (10 μg/ml) in the presence of either TNF or mSTAR (5 ng/ml). The response was measured at 72 hours by dilution of cell division dyes. Representative cell division plots are shown in FIG. 19.

FIG. 19—The TNFR2-specific agonist mSTAR can mimic TNF mediated CD4 T cell activation: Mouse CD4 T cells were activated via TCR with anti-CD3 (10 μg/ml) in the presence of TNF (5 ng/ml) or mSTAR (5 ng/ml). The response was measured at 72 hours by measuring total cell numbers. Data represent mean±S.E.M for triplicate samples.

FIG. 20—mSTAR can mimic TNF CD8 T cell activation: Mouse CD8 T cells were activated via TCR with anti-CD3 (10 μg/ml) in the presence of TNF (5 ng/ml), TNFR2 activation (mSTAR—5 ng/ml) with or without Smac-mimetic (500 μM). The response was measured at 72 hours by dilution of cell division dyes Representative cell division plots are shown in FIG. 21.

FIG. 21—mSTAR can mimic TNF CD8 T cell activation: Mouse CD8 T cells were activated via TCR with anti-CD3 (10 μg/ml) in the presence of TNF (5 ng/ml), TNFR2 activation (mSTAR—5 ng/ml) with or without Smac-mimetic (500 μM). The response was measured at 72 hours by measuring total cell numbers. Data represent mean±S.E.M for triplicate samples.

FIG. 22—mSTAR does not activate CD8 T cells in the absence of TNFR2: Naïve CD8 T cells isolated from WT or tnfr2−/− mice were activated via TCR with anti-CD3 (10 μg/ml) in the presence of TNF (5 ng/ml) or mSTAR (5 ng/ml) and the response measured at 72 hours by dilution of cell tracking dyes. Data are representative examples of triplicate cultures.

FIG. 23—TNF co-stimulation increases IFNγ production: Mouse CD8 T cells were activated via TCR with anti-CD3 (10 μg/ml) in the presence of soluble recombinant mouse TNF (5 ng/ml) with or without Smac-mimetic (500 μM) or IL-2 as described in Example 1. Supernatant was removed from T cell cultures at 48 hours and CBAs performed to measure IFNγ production. Data represent mean for duplicate samples. ND=none detected.

### KEY TO THE SEQUENCE LISTING

SEQ ID NO:1—human TNFR2 amino acid sequence

SEQ ID NO:2—hSTAR2 (also referred to as “TNC-scTNF(143N/145R)”) amino acid sequence

SEQ ID NO:3—mSTAR2 (also referred to as “TNC-sc-mTNF(221N/223R)”) amino acid sequence

SEQ ID NO:4—EHD2-sc-hTNF_{R2} amino acid sequence

SEQ ID NO:5—TNC-scTNF_{R2} amino acid sequence

SEQ ID NO:6—sc-mTNF_{R2} amino acid sequence

SEQ ID NO:7—p53-sc-mTNF_{R2} amino acid sequence

SEQ ID NO:8—GCN4-sc-mTNF_{R2} amino acid sequence

SEQ ID NO:9—TNF (D143N/A145R) amino acid sequence

SEQ ID NO:10—MHD2-scTNFR2 amino acid sequence

SEQ ID NO:11—EHD2-scTNFR2-L16aa amino acid sequence

SEQ ID NO:12—EHD2-scTNFR2-L28aa amino acid sequence

SEQ ID NO:13—sc-hTNF_{R2} amino acid sequence

SEQ ID NO:14—EHD2-sc-mTNF_{R2} amino acid sequence

human TNFR2 amino acid sequence

SEQ ID NO: 1

MAPVAVWAAL AVGLELWAAA HALPAQVAFT PYAPEPGSTC
RLREYYDQTA QMCCSKCSPG QHAKVFCTKT SDTVCDSCED
STYTQLWNWV PECLSCGSRC SSDQVETQAC TREQNRICTC
RPGWYCALSK QEGCRLCAPL RKCRPGFGVA RPGTETSDVV
CKPCAPGTFS NTTSSTDICR PHQICNVVAI PGNASMDAVC
TSTSPTRSMA PGAVHLPQPV STRSQHTQPT PEPSTAPSTS
FLLPMGPSPP AEGSTGDFAL PVGLIVGVTA LGLLIIGVVN
CVIMTQVKKK PLCLQREAKV PHLPADKARG TQGPEQQHLL
ITAPSSSSSS LESSASALDR RAPTRNQPQA PGVEASGAGE
ARASTGSSDS SPGGHGTQVN VTCIVNVCSS SDHSSQCSSQ
ASSTMGDTDS SPSESPKDEQ VPFSKEECAF RSQLETPETL
LGSTEEKPLP LGVPDAGMKP S
hSTAR2 (also referred to as “TNC-scTNF
(143N/145R)”) amino acid sequence

SEQ ID NO: 2

ACGCAAAPDI KDLLSRLEEL EGLVSSLREQ GGGGSVRSSS
RTPSDKPVAH VVANPQAEGQ LQWLNRRANA LLANGVELRD
NQLVVPSEGL YLIYSQVLFK GQGCPSTHVL LTHTISRIAV
SYQTKVNLLS AIKSPCQRET PEGAEAKPWY EPIYLGGVFQ
LEKGDRLSAE INRPDYLNFR ESGQVYFGII ALGGGGSVRS
SSRTPSDKPV AHVVANPQAE GQLQWLNRRA NALLANGVEL
RDNQLVVPSE GLYLIYSQVL FKGQGCPSTH VLLTHTISRI
AVSYQTKVNL LSAIKSPCQR ETPEGAEAKP WYEPIYLGGV
FQLEKGDRLS AEINRPDYLN FRESGQVYFG IIALGGGGSR
SSSRTPSDKP VAHVVANPQA EGQLQWLNRR ANALLANGVE
LRDNQLVVPS EGLYLIYSQV LFKGQGCPST HVLLTHTISR
IAVSYQTKVN LLSAIKSPCQ RETPEGAEAK PWYEPIYLGG
VFQLEKGDRL SAEINRPDYL NFRESGQVYF GIIAL
mSTAR2 (also referred to as “TNC-sc-mTNF
(221N/223R)”) amino acid sequence

SEQ ID NO: 3

ACGCAAAPDI KDLLSRLEEL EGLVSSLREQ GGGGSLRSSS
QNSSDKPVAH VVANHQVEEQ LEWLSQRANA LLANGMDLKD
NQLVVPADGL YLVYSQVLFK GQGCPDYVLL THTVSRFAIS
YQEKVNLLSA VKSPCPKDTP EGAELKPWYE PIYLGGVFQL
EKGDQLSAEV NLPKYLNFRE SGQVYFGVIA LGGGGSLRSS
SQNSSDKPVA HVVANHQVEE QLEWLSQRAN ALLANGMDLK
DNQLVVPADG LYLVYSQVLF KGQGCPDYVL LTHTVSRFAI
SYQEKVNLLS AVKSPCPKDT PEGAELKPWY EPIYLGGVFQ
LEKGDQLSAE VNLPKYLNFR ESGQVYFGVI ALGGGGSLRS
SSQNSSDKPV AHVVANHQVE EQLEWLSQRA NALLANGMDL
KDNQLVVPAD GLYLVYSQVL FKGQGCPDYV LLTHTVSRFA
ISYQEKVNLL SAVKSPCPKD TPEGAELKPW YEPIYLGGVF
QLEKGDQLSA EVNLPKYLNF RESGQVYFGV IAL
EHD2-sc-hTNFR2 amino acid sequence

SEQ ID NO: 4

METDTLLLWV LLLWVPGSTG DAAQPAGGGA AAHHHHHHGG
TGGGGSGGKL GGSGGDFTPP TVKILQSSCD GGGHFPPTIQ
LLCLVSGYTP GTINITWLED GQVMDVDLST ASTTQEGELA
STQSELTLSQ KHWLSDRTYT CQVTYQGHTF EDSTKKCADS
NGGGSGGGTG SEFLASSVRS SSRTPSDKPV AHVVANPQAE
GQLQWLNRRA NALLANGVEL RDNQLVVPSE GLYLIYSQVL
FKGQGCPSTH VLLTHTISRI AVSYQTKVNL LSAIKSPCQR
ETPEGAEAKP WYEPIYLGGV FQLEKGDRLS AEINRPDYLN
FRESGQVYFG IIALGGGGSV RSSSRTPSDK PVAHVVANPQ
AEGQLQWLNR RANALLANGV ELRDNQLVVP SEGLYLIYSQ
VLFKGQGCPS THVLLTHTIS RIAVSYQTKV NLLSAIKSPC
QRETPEGAEA KPWYEPIYLG GVFQLEKGDR LSAEINRPDY
LNFRESGQVY FGIIALGGGG SVRSSSRTPS DKPVAHVVAN
PQAEGQLQWL NRRANALLAN GVELRDNQLV VPSEGLYLIY
SQVLFKGQGC PSTHVLLTHT ISRIAVSYQT KVNLLSAIKS
PCQRETPEGA EAKPWYEPIY LGGVFQLEKG DRLSAEINRP
DYLNFRESGQ VYFGIIAL
TNC-scTNFR2 amino acid sequence

SEQ ID NO: 5

ACGCAAAPDV KELLSRLEEL ENLVSSLREQ GGGGSVRSSS
RTPSDKPVAH VVANPQAEGQ LQWLNRRANA LLANGVELRD
NQLVVPSEGL YLIYSQVLFK GQGCPSTHVL LTHTISRIAV
SYQTKVNLLS AIKSPCQRET PEGAEAKPWY EPIYLGGVFQ
LEKGDRLSAE INRPDYLNFR ESGQVYFGII ALGGGGSVRS
SSRTPSDKPV AHVVANPQAE GQLQWLNRRA NALLANGVEL
RDNQLVVPSE GLYLIYSQVL FKGQGCPSTH VLLTHTISRI
AVSYQTKVNL LSAIKSPCQR ETPEGAEAKP WYEPIYLGGV
FQLEKGDRLS AEINRPDYLN FRESGQVYFG IIALGGGGSV
RSSSRTPSDK PVAHVVANPQ AEGQLQWLNR RANALLANGV
ELRDNQLVVP SEGLYLIYSQ VLFKGQGCPS THVLLTHTIS
RIAVSYQTKV NLLSAIKSPC QRETPEGAEA KPWYEPIYLG
GVFQLEKGDR LSAEINRPDY LNFRESGQVY FGIIAL
sc-mTNFR2 amino acid sequence

SEQ ID NO: 6

LRSSSQNSSD KPVAHVVANH QVEEQLEWLS QRANALLANG
MDLKDNQLVV PADGLYLVYS QVLFKGQGCP DYVLLTHTVS
RFAISYQEKV NLLSAVKSPC PKDTPEGAEL KPWYEPIYLG
GVFQLEKGDQ LSAEVNLPKY LNFRESGQVY FGVIAL
p53-sc-mTNFR2 amino acid sequence

SEQ ID NO: 7

KKPLDGEYFT LQIRGRERFE MFRELNEALE LKDAQAGKEP
LRSSSQNSSD KPVAHVVANH QVEEQLEWLS QRANALLANG
MDLKDNQLVV PADGLYLVYS QVLFKGQGCP DYVLLTHTVS
RFAISYQEKV NLLSAVKSPC PKDTPEGAEL KPWYEPIYLG
GVFQLEKGDQ LSAEVNLPKY LNFRESGQVY FGVIALGGGG
SLRSSSQNSS DKPVAHVVAN HQVEEQLEWL SQRANALLAN
GMDLKDNQLV VPADGLYLVY SQVLFKGQGC PDYVLLTHTV
SRFAISYQEK VNLLSAVKSP CPKDTPEGAE LKPWYEPIYL
GGVFQLEKGD QLSAEVNLPK YLNFRESGQV YFGVIALGGG
GSLRSSSQNS SDKPVAHVVA NHQVEEQLEW LSQRANALLA
NGMDLKDNQL VVPADGLYLV YSQVLFKGQG CPDYVLLTHT
VSRFAISYQE KVNLLSAVKS PCPKDTPEGA ELKPWYEPIY
LGGVFQLEKG DQLSAEVNLP KYLNFRESGQ VYFGVIALGG
GGSLRSSSQN SSDKPVAHVV ANHQVEEQLE WLSQRANALL
ANGMDLKDNQ LVVPADGLYL VYSQVLFKGQ GCPDYVLLTH
TVSRFAISYQ EKVNLLSAVK SPCPKDTPEG AELKPWYEPI
YLGGVFQLEK GDQLSAEVNL PKYLNFRESG QVYFGVIAL
GCN4-sc-mTNFR2 amino acid sequence

SEQ ID NO: 8

RMKQLEDKVE ELLSKNYHLE NEVARLKKLV GERLRSSSQN
SSDKPVAHVV ANHQVEEQLE WLSQRANALL ANGMDLKDNQ
LVVPADGLYL VYSQVLFKGQ GCPDYVLLTH TVSRFAISYQ
EKVNLLSAVK SPCPKDTPEG AELKPWYEPI YLGGVFQLEK
GDQLSAEVNL PKYLNFRESG QVYFGVIALG GGGSLRSSSQ
NSSDKPVAHV VANHQVEEQL EWLSQRANAL LANGMDLKDN
QLVVPADGLY LVYSQVLFKG QGCPDYVLLT HTVSRFAISY
QEKVNLLSAV KSPCPKDTPE GAELKPWYEP IYLGGVFQLE
KGDQLSAEVN LPKYLNFRES GQVYFGVIAL GGGGSLRSSS
QNSSDKPVAH VVANHQVEEQ LEWLSQRANA LLANGMDLKD
NQLVVPADGL YLVYSQVLFK GQGCPDYVLL THTVSRFAIS
YQEKVNLLSA VKSPCPKDTP EGAELKPWYE PIYLGGVFQL
EKGDQLSAEV NLPKYLNFRE SGQVYFGVIA LGGGGSLRSS
SQNSSDKPVA HVVANHQVEE QLEWLSQRAN ALLANGMDLK
DNQLVVPADG LYLVYSQVLF KGQGCPDYVL LTHTVSRFAI
SYQEKVNLLS AVKSPCPKDT PEGAELKPWY EPIYLGGVFQ
LEKGDQLSAE VNLPKYLNFR ESGQVYFGVI ALGGGGSLRS
SSQNSSDKPV AHVVANHQVE EQLEWLSQRA NALLANGMDL
KDNQLVVPAD GLYLVYSQVL FKGQGCPDYV LLTHTVSRFA
ISYQEKVNLL SAVKSPCPKD TPEGAELKPW YEPIYLGGVF
QLEKGDQLSA EVNLPKYLNF RESGQVYFGV IAL
TNF (D143N/A145R) amino acid sequence

SEQ ID NO: 9

VRSSSRTPSD KPVAHVVANP QAEGQLQWLN RRANALLANG
VELRDNQLVV PSEGLYLIYS QVLFKGQGCP STHVLLTHTI
SRIAVSYQTK VNLLSAIKSP CQRETPEGAE AKPWYEPIYL
GGVFQLEKGD RLSAEINRPD YLNFRESGQV YFGIIAL
MHD2-scTNFR2 amino acid sequence

SEQ ID NO: 10

METDTLLLWV LLLWVPGSTG DAAQPAGGGA AAHHHHHHGG
TGGGGSGGKL GGSGGAELPP KVSVFVPPRD GFFGNPRKSK
LICQATGFSP RQIQVSWLRE GKQVGSGVTT DQVQAEAKES
GPTTYKVTST LTIKESDWLG QSMFTCRVDH RGLTFQQNAS
SMCVPDGGGS GGGTGSEFLA SSRTPSDKPV AHVVANPQAE
GQLQWLNRRA NALLANGVEL RDNQLVVPSE GLYLIYSQVL
FKGQGCPSTH VLLTHTISRI AVSYQTKVNL LSAIKSPCQR
ETPEGAEAKP WYEPIYLGGV FQLEKGDRLS AEINRPDYLN
FRESGQVYFG IIALGGGGSS SRTPSDKPVA HVVANPQAEG
QLQWLNRRAN ALLANGVELR DNQLVVPSEG LYLIYSQVLF
KGQGCPSTHV LLTHTISRIA VSYQTKVNLL SAIKSPCQRE
TPEGAEAKPW YEPIYLGGVF QLEKGDRLSA EINRPDYLNF
RESGQVYFGI IALGGGGSSS RTPSDKPVAH VVANPQAEGQ
LQWLNRRANA LLANGVELRD NQLVVPSEGL YLIYSQVLFK
GQGCPSTHVL LTHTISRIAV SYQTKVNLLS AIKSPCQRET
PEGAEAKPWY EPIYLGGVFQ LEKGDRLSAE INRPDYLNFR
ESGQVYFGII AL
EHD2-scTNFR2-L16aa amino acid sequence

SEQ ID NO: 11

METDTLLLWV LLLWVPGSTG DAAQPAGGGA AAHHHHHHGG
TGGGGSGGKL GGSGGDFTPP TVKILQSSCD GGGHFPPTIQ
LLCLVSGYTP GTINITWLED GQVMDVDLST ASTTQEGELA
STQSELTLSQ KHWLSDRTYT CQVTYQGHTF EDSTKKCADS
NGGGSGGGTG SEFLASSRTP SDKPVAHVVA NPQAEGQLQW
LNRRANALLA NGVELRDNQL VVPSEGLYLI YSQVLFKGQG
CPSTHVLLTH TISRIAVSYQ TKVNLLSAIK SPCQRETPEG
AEAKPWYEPI YLGGVFQLEK GDRLSAEINR PDYLNFRESG
QVYFGIIALG GGGSSSRTPS DKPVAHVVAN PQAEGQLQWL
NRRANALLAN GVELRDNQLV VPSEGLYLIY SQVLFKGQGC
PSTHVLLTHT ISRIAVSYQT KVNLLSAIKS PCQRETPEGA
EAKPWYEPIY LGGVFQLEKG DRLSAEINRP DYLNFRESGQ
VYFGIIALGG GGSSSRTPSD KPVAHVVANP QAEGQLQWLN
RRANALLANG VELRDNQLVV PSEGLYLIYS QVLFKGQGCP
STHVLLTHTI SRIAVSYQTK VNLLSAIKSP CQRETPEGAE
AKPWYEPIYL GGVFQLEKGD RLSAEINRPD YLNFRESGQV
YFGIIAL
EHD2-scTNFR2-L28aa amino acid sequence

SEQ ID NO: 12

METDTLLLWV LLLWVPGSTG DAAQPAGGGA AAHHHHHHGG
TGGGGSGGKL GGSGGDFTPP TVKILQSSCD GGGHFPPTIQ
LLCLVSGYTP GTINITWLED GQVMDVDLST ASTTQEGELA
STQSELTLSQ KHWLSDRTYT CQVTYQGHTF EDSTKKCADS
NGGGSGGGSG GGSGGGSGGG SGGSEFLASS RTPSDKPVAH
VVANPQAEGQ LQWLNRRANA LLANGVELRD NQLVVPSEGL
YLIYSQVLFK GQGCPSTHVL LTHTISRIAV SYQTKVNLLS
AIKSPCQRET PEGAEAKPWY EPIYLGGVFQ LEKGDRLSAE
INRPDYLNFR ESGQVYFGII ALGGGGSSSR TPSDKPVAHV
VANPQAEGQL QWLNRRANAL LANGVELRDN QLVVPSEGLY
LIYSQVLFKG QGCPSTHVLL THTISRIAVS YQTKVNLLSA
IKSPCQRETP EGAEAKPWYE PIYLGGVFQL EKGDRLSAEI
NRPDYLNFRE SGQVYFGIIA LGGGGSSSRT PSDKPVAHVV
ANPQAEGQLQ WLNRRANALL ANGVELRDNQ LVVPSEGLYL
IYSQVLFKGQ GCPSTHVLLT HTISRIAVSY QTKVNLLSAI
KSPCQRETPE GAEAKPWYEP IYLGGVFQLE KGDRLSAEIN
RPDYLNFRES GQVYFGIIAL
sc-hTNFR2 amino acid sequence

SEQ ID NO: 13

VRSSSRTPSD KPVAHVVANP QAEGQLQWLN RRANALLANG
VELRDNQLVV PSEGLYLIYS QVLFKGQGCP STHVLLTHTI
SRIAVSYQTK VNLLSAIKSP CQRETPEGAE AKPWYEPIYL
GGVFQLEKGD RLSAEINRPD YLNFRESGQV YFGIIAL
EHD2-sc-mTNFR2 amino acid sequence

SEQ ID NO: 14

METDTLLLWV LLLWVPGSTG DAAQPAGGGA AAHHHHHHGG
TGGGGSGGKL GGSGGDFTPP TVKILQSSCD GGGHFPPTIQ
LLCLVSGYTP GTINITWLED GQVMDVDLST ASTTQEGELA
STQSELTLSQ KHWLSDRTYT CQVTYQGHTF EDSTKKCADS
NGGGSGGGTG SEFLASSLRS SSQNSSDKPV AHVVANHQVE
EQLEWLSQRA NALLANGMDL KDNQLVVPAD GLYLVYSQVL
FKGQGCPDYV LLTHTVSRFA ISYQEKVNLL SAVKSPCPKD
TPEGAELKPW YEPIYLGGVF QLEKGDQLSA EVNLPKYLNF
RESGQVYFGV IALGGGGSLR SSSQNSSDKP VAHVVANHQV
EEQLEWLSQR ANALLANGMD LKDNQLVVPA DGLYLVYSQV
LFKGQGCPDY VLLTHTVSRF AISYQEKVNL LSAVKSPCPK
DTPEGAELKP WYEPIYLGGV FQLEKGDQLS AEVNLPKYLN
FRESGQVYFG VIALGGGGSL RSSSQNSSDK PVAHVVANHQ
VEEQLEWLSQ RANALLANGM DLKDNQLVVP ADGLYLVYSQ
VLFKGQGCPD YVLLTHTVSR FAISYQEKVN LLSAVKSPCP
KDTPEGAELK PWYEPIYLGG VFQLEKGDQL SAEVNLPKYL
NFRESGQVYF GVIAL

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in immunology, molecular biology, cancer therapy, pharmacology, protein chemistry, and biochemistry).

Unless otherwise indicated, the techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al., (editors) Current Protocols in Immunology, John Wiley & Sons (including all updates until present).

As used herein, the term about, unless stated to the contrary, refers to +/−10%, more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

The term “effector T cell” refers to a subset of T cells that actively respond to a stimulus, including Y cell receptor signaling or co-stimulation, to direct an immune response against cells displaying their corresponding antigen, through secretion of cytokines or cytotoxins, for example. This includes effector CD4⁺FoxP3− (“helper”) and CD8⁺ (“killer”) T cell types. As used herein, the term “effector T cell” does not encompass regulatory T cells (“T_{reg}s”) which act to supress activation and proliferation of effector T cells.

The term “TNFR2” refers to tumour necrosis factor receptor 2, also known as tumour necrosis factor receptor superfamily member 1B (TNFRSF1B) and CD120b. TNFR2 is a membrane-bound receptor that binds to tumour necrosis factor-alpha (TNFα). The amino acid sequence of human TNFR2 (including its signal peptide) is provided in SEQ ID NO: 1.

The term “TNFR2-specific agonist” refers to any agent that can specifically increase signaling activity mediated by TNFR2. By “specific” it is meant that the agent preferentially activates TNFR2 relative to TNFR1, i.e., at a given concentration, the TNFR2-specific agonist increases TNFR2-mediated signaling to a greater degree than it increases TNFR1-mediated signaling. For the avoidance of doubt, TNFα is not a TNFR2-specific agonist, in the context of the present disclosure, because it preferentially activates TNFR1 signaling relative to TNFR2. In some embodiments, the TNFR2-specific agonist binds to TNFR2 with an affinity which is at least 1.2-fold, at least 1.5-fold, at least 2-fold, at least 5-fold, or at least 10-fold stronger than its affinity for binding to TNFR1. In some embodiments, the TNFR2-specific agonist does not detectably bind TNFR1. In some embodiments, the TNFR2-specific agonist activates TNFR2 signaling at a level which is at least 1.2-fold, at least 1.5-fold, at least 2-fold, at least 5-fold, or at least 10-fold higher than it activates TNFR1 signaling. In some embodiments, the TNFR2-specific agonist does not activate a detectable level of TNFR1 signaling. In some embodiments, the TNFR2-specific antagonist does not inhibit TNFR1 signaling by a detectable amount.

The term “TNFR2-specific antagonist” as used herein, refers to any agent that can specifically reduce signaling activity mediated by TNFR2. By “specific” it is meant that the agent preferentially inhibits TNFR2 relative to TNFR1, i.e., at a given concentration, the TNFR2-specific antagonist inhibits TNFR2-mediated signaling to a greater degree than it inhibits TNFR1-mediated signaling. In some embodiments, the TNFR2-specific antagonist binds to TNFR2 with an affinity which is at least 1.2-fold, at least 1.5-fold, at least 2-fold, at least 5-fold, or at least 10-fold stronger than its affinity for binding to TNFR1. In some embodiments, the TNFR2-specific antagonist does not detectably bind TNFR1. In some embodiments, the TNFR2-specific antagonist inhibits TNFR2 signaling at a level of inhibition which is at least at least 1.2-fold, 1.5-fold, at least 2-fold, at least 5-fold, or at least 10-fold higher than it inhibits TNFR1 signaling.

As used herein, “competitive inhibition” refers to a mode of inhibition of a target molecule in which an inhibitor (i.e., a “competitive inhibitor”) binds to a functionally critical site on a target molecule itself (e.g., a ligand binding site) or on a ligand (e.g., a binding partner molecule) for the target molecule thereby sterically hindering interaction of the target molecule with the ligand. The competitive inhibitor may, but does not necessarily, have a higher affinity for the target molecule than the molecule with which it competes (e.g., the ligand).

The term “polypeptide” or “protein” as used herein, refer to a polymer of amino acids generally greater than about 20 amino acids in length.

As used herein, the term “binds” is in reference to a detectable interaction between two molecules, for example, between an inhibitor and its target. As used herein, the term “specifically binds” or “binds specifically”, or variations thereof, shall be taken to mean that a binding molecule associates more frequently, more rapidly, with greater duration and/or with greater affinity with a particular molecule than it does with alternative molecules. Generally, but not necessarily, reference to binding means specific binding, and each term shall be understood to provide explicit support for the other term.

As used herein, the term “subject” can be any animal. In one embodiment, the animal is a vertebrate. For example, the animal can be a mammal, avian, chordate, amphibian or reptile. Exemplary subjects include but are not limited to human, primate, livestock (e.g. sheep, cow, chicken, horse, donkey, pig), companion animals (e.g. dogs, cats), laboratory test animals (e.g. mice, rabbits, rats, guinea pigs, hamsters), captive wild animal (e.g. fox, deer). In one embodiment, the mammal is a human.

As the skilled person would understand, the TNFR2-specific agonists and antagonists described herein will be administered in a therapeutically effective amount. The terms “effective amount” or “therapeutically effective amount” as used herein, refer to a sufficient amount of an agonist or antagonist being administered which will relieve to some extent or prevent worsening of one or more of the symptoms of the disease or condition being treated. The result can be reduction or a prevention of progression of the signs, symptoms, or causes of a disease, or any other desired alteration of a biological system. For example, an “effective amount” for therapeutic uses is the amount of the agonist or antagonist required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of an agonist or antagonist is an amount effective to achieve a desired pharmacologic effect or therapeutic improvement without undue adverse side effects. It is understood that “an effective amount” or “a therapeutically effective amount” can vary from subject to subject, due to variation in metabolism of the compound of any of age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial. Where more than one therapeutic agent is used in combination, a “therapeutically effective amount” of each therapeutic agent can refer to an amount of the therapeutic agent that would be therapeutically effective when used on its own, or may refer to a reduced amount that is therapeutically effective by virtue of its combination with one or more additional therapeutic agents.

The term “small molecule” as used herein, refers to a molecule having a molecular weight below about 2000 daltons.

The terms “treating” or “treatment” as used herein, refer to both direct treatment of a subject by a medical professional (e.g., by administering a therapeutic agent to the subject), or indirect treatment, effected, by at least one party, (e.g., a medical doctor, a nurse, a pharmacist, or a pharmaceutical sales representative) by providing instructions, in any form, that (i) instruct a subject to self-treat according to a claimed method (e.g., self-administer a drug) or (ii) instruct a third party to treat a subject according to a claimed method. Also encompassed within the meaning of the term “treating” or “treatment” are prevention or reduction of the disease to be treated, e.g., by administering a therapeutic at a sufficiently early phase of disease to prevent or slow its progression.

The terms “co-administration” or “administered in combination” or the like, as used herein, are meant to encompass administration of the selected therapeutic agents to a single subject, and are intended to include treatment regimens in which the agents are administered by the same or different route of administration or at the same or different time.

### T Cell Activation

Activation of effector T cells occurs through the simultaneous engagement of the T cell receptor and a co-stimulatory molecule (like CD28, or ICOS) on the T cell by the major histocompatibility complex (MHCII) peptide and co-stimulatory molecules on an antigen presenting cell. Both are required for production of an effective immune response; in the absence of co-stimulation, T cell receptor signaling alone results in anergy. The signaling pathways downstream from co-stimulatory molecules usually engages the PI3K pathway generating PIP3 at the plasma membrane and recruiting PH domain containing signaling molecules like PDK1 that are essential for the activation of PKCθ, and eventual IL-2 production.

The first signal is provided by binding of the T cell receptor to its cognate peptide presented on MHCII on an APC. The second signal comes from co-stimulation, in which surface receptors on the T cell are induced by a number of stimuli, including products of pathogens, but sometimes breakdown products of cells, such as necrotic-bodies or heat shock proteins. A canonical co-stimulatory receptor expressed constitutively by naïve effector T cells is CD28, co-stimulation for these cells can come from the CD80 and CD86 proteins, which together constitute the B7 protein, (B7.1 and B7.2, respectively) on the APC for example. Other co-stimulatory receptors are expressed upon activation of the T cell, such as OX40 and ICOS. The co-stimulatory signal, in combination with TCR signaling activates the T cell to respond to an antigen leading to proliferation and cytokine production.

The present inventors have surprisingly found that TNFR2 is a potent co-stimulatory receptor for activation of effector T cells. Thus, TNFR2 provides a target for selectively increasing or inhibiting effector T cell activation in a subject. For example, it may be advantageous to increase effector T cell activation in a subject which has a disease, such as cancer or an infection, which would benefit from an increase in effector T cell activation. Conversely, over-activation of effector T cells, for example in autoimmune disease, can be inhibited by reducing TNFR2 signaling.

Similarly, TNFR2 signaling can be used to increase activation of effector T cells in vitro. Such methods can be used to expand a population of effector T cells in cell culture, for example.

Levels of effector T cell activation can be measured by any method known in the art, including the methods described in the Examples or in Caruso et al. (1997), Bercovici et al. (2000), and Plebanski et al. (2010), for example. Such methods typically involve quantifying the amount of effector T cells in a sample, thereby measuring cell proliferation, or by quantifying the amount of cytokine production in a sample. Routine techniques such as flow cytometry and enzyme-linked immunosorbent assay (ELISA) and enzyme-linked immunospot (ELISPOT) are suitable for assessing levels of T cell activation, as well as limiting dilutions culture, intracellular staining, cytokine capture, tetramer staining and spectratyping and biosensor assays.

### TNFR2 Signaling

The present invention involves specifically modulating TNFR2 signaling to either increase or inhibit T cell activation. This is achieved through the use of TNFR2-specific agonists or antagonists, which preferentially activate or inhibit TNFR2 signaling relative to TNFR1 signaling respectively.

TNFR2 signaling can be measured by any method known in the art. For example, suitable methods include determining the levels of endothelial/epithelial tyrosine kinase (Etk) expression and/or phosphorylation. Etk (also known as Bmx; bone marrow tyrosine kinase in chromosome X) is a kinase which is activated by TNFR2 and has been implicated in cell adhesion, migration, proliferation, and survival (Tamagnone et al., 1994; Abassi et al., 2003). In epithelial cells, Etk may be a regulator of cell junctions (Hamm-Alvarez et al., 2001). In vascular endothelial cells (EC), Etk is involved in TNF-induced angiogenic events (Zhang et al., 2003; Pan et al., 2002) and mediates activation of the phosphatidylinositol 3 kinase (PI3K)-Atk angiogenic pathway, which is involved in growth factor stimulated cell migration (Kureishi et al., 2000). The amino acid sequence of Etk has the database accession number P51813 GI: 1705489.

The expression and phosphorylation of Etk in ECs is indicative of TNFR2 signaling. Thus, TNFR2 signaling can be measured by determining the level of expression or phosphorylation of Etk in ECs in a sample from a subject. Phosphorylation of Etk may be determined at Tyr566. Increased phosphorylation of Tyr 566 relative to controls may be indicative of TNFR2 activation. In some embodiments, phosphorylation of Etk may be determined in tubular epithelial cells (TEC) of the sample.

Activation of TNFR1 signaling, on the other hand, may be determined by any convenient method known in the art. Suitable methods include, for example, determining the phosphorylation of ASK1 at residue Thr845 and/or the absence of phosphorylation at residue Ser966, determining the up-regulation of E-selectin in endothelial cells (Slowik et al., 1993) and determining the apoptosis of cells, for example kidney cells, expressing the TNFR1 polypeptide. Generally, increased phosphorylation of Thr845 and/or decreased phosphorylation of Ser966 relative to controls may be indicative of TNFR1 activation. In some embodiments, phosphorylation of ASK1 may be determined in endothelial cells (EC) in a sample from a subject. The amino acid sequence of ASK1 has the database accession number BAA12684.1 GI: 1805500.

The level of Etk expression and/or the phosphorylation of ASK1 and/or Etk may be determined by standard immunological techniques. For example, the sample may be contacted with an antibody that binds specifically to the target molecule to be detected (i.e. Etk, phosphorylated Etk or phosphorylated ASK1) and the binding of the antibody to the sample determined.

An antibody which specifically binds to an antigen such as Etk, phosphorylated Etk or phosphorylated ASK1 may not show any significant binding to molecules in mammalian cells other than the antigen. An antibody that specifically binds to Etk, phosphorylated Etk or phosphorylated ASK1 may be generated using techniques which are conventional in the art as described above.

Samples to be subjected to contact with an antibody may be prepared using any available technique that allows the antibody to bind to bind to cellular polypeptides in the sample.

Binding of the antibody to the sample may be determined by any appropriate means. Tagging with individual reporter molecules is one possibility. A reporter molecule may be linked to the primary antibody that binds to the target molecule or to a secondary antibody that binds to the primary antibody. The reporter molecule may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding binding molecule (e.g. antibody) and reporter molecule. One favoured mode is by covalent linkage of a binding member with an individual fluorochrome, phosphor or laser dye with spectrally isolated absorption or emission characteristics. Suitable fluorochromes include fluorescein, rhodamine, phycoerythrin and Texas Red. Suitable chromogenic dyes include diaminobenzidine. Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes that catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed. Further examples are horseradish peroxidase and chemiluminescence.

Other suitable methods for determining the levels of TNFR2 and TNFR1 signaling are described in U.S. Pat. No. 9,081,017.

### TNFR2-Specific Agonists

Examples of types of TNFR2-specific agonists useful for increasing effector T cell activation include, but are not limited to, a small molecule, a polynucleotide, a polypeptide, or a peptide.

### TNF Mutein Polypeptides

In some embodiments the TNFR2-specific agonist is a polypeptide, which may activate TNFR2-mediated signaling activity by at least one of a number of different mechanisms.

In some embodiments, the TNFR2-specific agonist is a TNFR2-specific TNF mutein. TNFR2-specific TNF muteins are mutant forms of TNFα which bind preferentially to TNFR2 relative to TNFR1. The structure and activity of wild type TNFα has been well-characterised in the art. The residues involved in TNF receptor binding are located at the base of the homotrimeric structure of TNFα at each side of the inter-subunit groove that separates two monomeric TNFα subunits (Van Ostade et al., 1991). Mutation of these receptor-binding residues may confer specificity for TNFR2 and the skilled person is readily able to produce and characterise suitable TNF mutein polypeptides with specificity for TNFR2 relative to TNFR1.

A suitable TNF mutein polypeptide which binds preferentially to TNFR2 relative to TNFR1 may comprise or consist of at least part of the wild-type TNFα sequence (NP 000585.2 GI: 25952111) or a variant thereof, with one or more mutations which increase TNFR2 binding relative to TNFR1 binding or, conversely, reduce TNFR1 binding relative to TNFR2 binding. Suitable mutations include non-conservative substitutions of the Asp residue at position 143, including for example, Asp143Tyr, Asp143Phe, or Asp143Asn (Van Ostade et al., 1994). A TNF mutein polypeptide may further include non-conservative substitutions of the Ala residue at position 145, for example Ala145Arg. Thus, in some embodiments, the TNFR2-specific TNF mutein comprises an amino acid substitution at Asp143 and/or Ala145. In some embodiments, the substitution is one or more of Asp143Tyr, Asp143Phe, Asp143Asn, or Ala145Arg. In one embodiment, the TNFR2-specific TNF mutein comprises the amino acid substitutions Asp143Asn and Ala145Arg.

Suitable TNFR2-specific TNF muteins include mSTAR2, hSTAR2, sc-mTNF_{R2}, EHD2-sc-hTNF_{R2}, p53-sc-mTNF_{R2}, GCN4-sc-mTNF_{R2}, TNC-scTNF_{R2}, or TNF (D143N/A145R), for example. Other suitable TNF muteins include MHD2-scTNFR2, EFID2-scTNFR2-L16aa, EHD2-scTNFR2-L28aa, sc-hTNF_{R2}, and EHD2-sc-mTNF_{R2}.

In some embodiments, the TNFR2-specific agonist is a polypeptide comprising a sequence which is at least 70% or at least 80% or at least 90% or at least 95% identical to SEQ ID NO:2 or SEQ ID NO:3 or a biologically active fragment thereof. SEQ ID NO:2 and SEQ ID NO:3 correspond to the amino acid sequences of hSTAR2 and mSTAR2 (“selective TNF-based agonist of TNF receptor 2” as described in Rauert et al., 2010 and Chopra et al., 2016) respectively. In some embodiments, the TNFR2-specific agonist is hSTAR2 or mSTAR2 (also referred to as “human STAR2” and “murine STAR2” respectively).

In some embodiments, the TNFR2-specific agonist is a polypeptide comprising a sequence which is at least 70% or at least 80% or at least 90% or at least 95% identical to SEQ ID NO:4 or a biologically active fragment thereof. SEQ ID NO:4 corresponds to the amino acid sequence of EHD2-sc-hTNF_{R2} described in Dong et al. (2016). Thus, in some embodiments, the TNFR2-specific agonist is EHD2-sc-hTNF_{R2}.

In some embodiments, the TNFR2-specific agonist is a polypeptide comprising a sequence which is at least 70% or at least 80% or at least 90% or at least 95% identical to SEQ ID NO:5 or a biologically active fragment thereof. SEQ ID NO:5 corresponds to TNC-scTNF_{R2} as described in Fischer et al. (2011). Thus, in some embodiments, the TNFR2-specific agonist is TNC-scTNF_{R2}.

Other suitable TNFR2-specific TNF muteins are described in US2008/0176796, US2015/0056159, U.S. Pat. Nos. 5,486,463, 5,422,104, WO86/02381, WO86/04606, WO88/06625, EP155549, EP168214, EP251037, EP340333, EP486908, Chopra et al. (2016), Fischer et al. (2011), Fischer et al. (2017), Dong et al. (2016), Rauert et al. (2010) and Loetscher et al. (1993).

### Polypeptides Comprising Antigen Binding Sites

In some embodiments, the TNFR2-specific agonist is a polypeptide comprising an antigen binding site such as an antibody, or a fragment thereof, which binds to TNFR2. Thus, in some embodiments, the TNFR2-specific agonist is an agonistic antibody. Such antibodies, or fragments thereof, may specifically bind to TNFR2 and activate TNFR2-mediated signaling. Suitable antibodies preferentially activate TNFR2 signaling relative to TNFR1 signaling. In some embodiments, the TNFR2-specific agonist antibody binds with higher affinity to TNFR2 relative to TNFR1.

The term “antibody” as used herein includes polyclonal antibodies, monoclonal antibodies, bispecific antibodies, fusion diabodies, triabodies, heteroconjugate antibodies, and chimeric antibodies. Also contemplated are antibody fragments that retain at least substantial (about 10%) antigen binding relative to the corresponding full length antibody. Such antibody fragments are referred to herein as “antigen-binding fragments” and comprise an antigen binding site of an antibody. Antibodies include modifications in a variety of forms including, for example, but not limited to, domain antibodies including either the VH or VL domain, a dimer of the heavy chain variable region (VHH, as described for a camelid), a dimer of the light chain variable region (VLL), Fv fragments containing only the light (VL) and heavy chain (VH) variable regions which may be joined directly or through a linker, or Fd fragments containing the heavy chain variable region and the CH1 domain.

A scFv consisting of the variable regions of the heavy and light chains linked together to form a single-chain antibody and oligomers of scFvs such as diabodies and triabodies are also encompassed by the term “antibody”. Also encompassed are fragments of antibodies such as Fab, (Fab′)2 and FabFc2 fragments which contain the variable regions and parts of the constant regions. Complementarity determining region (CDR)-grafted antibody fragments and oligomers of antibody fragments are also encompassed. The heavy and light chain components of an Fv may be derived from the same antibody or different antibodies thereby producing a chimeric Fv region. The antibody may be of animal (for example mouse, rabbit or rat) or human origin or may be chimeric or humanize.

As used herein the term “antibody” includes the various forms described herein. Using the guidelines provided herein and those methods well known to those skilled in the art which are described in the references cited above and in such publications as Harlow & Lane, Antibodies: a Laboratory Manual, Cold Spring Harbor Laboratory, (1988) the antibodies for use in the methods of the present invention can be readily made.

The antibodies may be Fv regions comprising a variable light (VL) and a variable heavy (VH) chain in which the light and heavy chains may be joined directly or through a linker. As used herein a linker refers to a molecule that is covalently linked to the light and heavy chain and provides enough spacing and flexibility between the two chains such that they are able to achieve a conformation in which they are capable of specifically binding the epitope to which they are directed. Protein linkers are particularly preferred as they may be expressed as an intrinsic component of the Ig portion of the fusion polypeptide.

In another embodiment, recombinantly produced single chain scFv antibody, preferably a humanized scFv, is used in the methods of the invention.

Methods for generating antibodies and fragments thereof are known in the art and/or described in Harlow and Lane (editors) Antibodies: A Laboratory Manual, Cold Spring Harbor Laboratory, (1988). Generally, in such methods, an antigen such as TNFR2 or a region thereof (e.g., an extracellular region of TNFR2) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

Monoclonal antibodies are one exemplary form of an antibody contemplated by the present disclosure. The term “monoclonal antibody” or “mAb” refers to a homogeneous antibody population capable of binding to the same antigen(s), for example, to the same epitope within the antigen. This term is not intended to be limited as regards to the source of the antibody or the manner in which it is made.

For the production of mAbs any one of a number of known techniques may be used, such as, for example, the procedure exemplified in U.S. Pat. No. 4,196,265.

Alternatively, ABL-MYC technology (NeoClone, Madison Wis. 53713, USA) is used to produce cell lines secreting MAbs (e.g., as described in Largaespada et al., 1996).

Antibodies can also be produced or isolated by screening a display library, e.g., a phage display library, e.g., as described in U.S. Pat. Nos. 6,300,064 and/or 5,885,793.

The antibody of the present disclosure may be a synthetic antibody. For example, the antibody is a chimeric antibody, a humanized antibody, a human antibody or a de-immunized antibody.

In one embodiment, an antibody described herein is a chimeric antibody. The term “chimeric antibody” refers to antibodies in which a portion of the heavy and/or light chain is identical with or homologous to corresponding sequences in antibodies derived from a particular species (e.g., murine, such as mouse) or belonging to a particular antibody class or subclass, while the remainder of the chain(s) is identical with or homologous to corresponding sequences in antibodies derived from another species (e.g., primate, such as human) or belonging to another antibody class or subclass. Methods for producing chimeric antibodies are described in, e.g., U.S. Pat. Nos. 4,816,567 and 5,807,715.

The antibodies of the present disclosure may be humanized or human.

The term “humanized antibody” shall be understood to refer to a subclass of chimeric antibodies having an antigen binding site or variable region derived from an antibody from a non-human species and the remaining antibody structure based upon the structure and/or sequence of a human antibody. In a humanized antibody, the antigen-binding site generally comprises the complementarity determining regions (CDRs) from the non-human antibody grafted onto appropriate FRs in the variable regions of a human antibody and the remaining regions from a human antibody. Antigen binding sites may be wild-type (i.e., identical to those of the non-human antibody) or modified by one or more amino acid substitutions. In some instances, FR residues of the human antibody are replaced by corresponding non-human residues.

Methods for humanizing non-human antibodies or parts thereof (e.g., variable regions) are known in the art. Humanization can be performed following the method of U.S. Pat. Nos. 5,225,539 or 5,585,089. Other methods for humanizing an antibody are not excluded.

The TNFR2-specific agonist can be a monoclonal antibody that binds TNFR2, such as MR2-1 (Hycult) or MAB2261 (R&D Systems, Inc.). Other suitable anti-TNFR2 antibodies that are capable of acting as TNFR2 agonists are described in Grell et al. (1995), Galloway et al. (1992), He et al. (2016), Tartaglia et al. (1993), Smith et al. (1994), Amrani et al. (1996), Okubo et al. (2013 and 2016), and Tam et al. (2019).

In some embodiments, the TNFR2-specific agonist is MR2-1, MAB2261, or a fragment of any one thereof. In some embodiments, the TNFR2-specific agonist is an antibody described in Grell et al. (1995), Galloway et al. (1992), He et al. (2016), Tartaglia et al. (1993), Smith et al. (1994), Amrani et al. (1996), and Okubo et al. (2013 and 2016), or a fragment of any one thereof. In some embodiments, the TNFR2-specific agonist comprises the CDRs of MR2-1 or MAB2261. In some embodiments, the TNFR2-specific agonist comprises the CDRs of an antibody described in Grell et al. (1995), Galloway et al. (1992), He et al. (2016), Tartaglia et al. (1993), Smith et al. (1994), Amrani et al. (1996) or Okubo et al. (2013 and 2016).

In some embodiments, the TNFR2-specific agonist is an antibody described in Tam et al. (2019). For instance, in some embodiments, the TNFR2-specific agonist is Y7, Y9, Y10, M3, H5L10, Abl, or Ab2, as described in Tam et al. (2019). In some embodiments, the TNFR2-specific agonist comprises the CDRs of an antibody described in Tam et al. (2019). In some embodiments, the TNFR2-specific agonist comprises the CDRs of Y7, Y9, Y10, M3, H5L10, Ab1, or Ab2, as described in Tam et al. (2019).

### Peptides

In some embodiments, the TNFR2-specific agonist is a peptide. Such peptides can be identified using techniques known in the art such as high throughput screens. Candidate peptides can be assessed for their ability to preferentially activate TNFR2 signaling relative to TNFR1 signaling using methods described herein. Peptides that are capable of acting as a TNFR2-specific agonist can include an 11 amino acid TNF receptor agonist peptide (TNF₇₀₋₈₀) described in Laichalk et al. (1998).

### Polynucleotides Encoding Peptides or Polypeptides

In some embodiments the TNFR2-specific agonist is a polynucleotide, which may activate TNFR2-mediated signaling activity by at least one of a number of different mechanisms.

In some embodiments, a polynucleotide-based TNFR2-specific agonist encodes a polypeptide, so that delivery of the polynucleotide to cells results in expression of an encoded peptide or polypeptide TNFR2-specific agonist. For example, the polynucleotide may encode any one or more of the polypeptide or peptide TNFR2-specific agonists described above.

In some embodiments, the polynucleotide TNFR2-specific agonist is provided in an expression vector to be delivered to cells (e.g., neurons) using any of a number of routine targeting methods known in the art.

As used herein, an “expression vector” is a DNA or RNA vector that is capable of effecting expression of one or more polynucleotides in a host cell (e.g., a neuron). The vector is typically a plasmid or recombinant virus. Any suitable expression vector can be used, examples of which include, but are not limited to, a plasmid or viral vector. In some embodiments, the viral vector is a retrovirus, a lentivirus, an adenovirus, a herpes virus, or an adeno-associated viral vector.

Such vectors will include one or more promoters for expressing the polynucleotide such as a dsRNA for gene silencing. Suitable promoters include include, but are not limited to, the retroviral LTR; the SV40 promoter; and the human cytomegalovirus (CMV) promoter. Cellular promoters such as eukaryotic cellular promoters including, but not limited to, the histone, RNA polymerase III (in the case of snRNA or miRNA expression), and β-actin promoters, can also be used. In some embodiments the promoter is an effector T cell-selective promoter. The selection of a suitable promoter will be apparent to those skilled in the art from the teachings contained herein.

### Small Molecules

In some embodiments, the TNFR2-specific agonist is a small molecule. In some embodiments, the small molecule binds to TNFR2 and increases it signaling activity. Suitable small molecule TNFR2-specific agonists for use in the invention can be identified using screening methods that are routine in the art.

In some embodiments, the small molecule that is administered may be a precursor compound, commonly referred to as a “prodrug” which is inactive or comparatively poorly active, but which, following administration, is converted (i.e., metabolised) to an active TNFR2-specific agonist. In those embodiments, the compound that is administered may be referred to as a prodrug. Alternatively, or in addition, the compounds that are administered may be metabolized to produce active metabolites which have activity in increasing TNFR2-mediated signaling activity. The use of such active metabolites is also within the scope of the present disclosure.

Depending on the substituents present in the compound, the compound may optionally be present in the form of a pharmaceutically acceptable salt. Salts of compounds which are suitable for use in the described methods are those in which a counter-ion is pharmaceutically acceptable. Suitable salts include those formed with organic or inorganic acids or bases. In particular, suitable salts formed with acids include those formed with mineral acids, strong organic carboxylic acids, such as alkane carboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁₋₄)-alkyl- or aryl-sulfonic acids which are substituted or unsubstituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidien, pyrrolidine, a mono-, di- or tri-lower alkylamine, for example ethyl-, tbutyl-, diethyl-, diisopropyl-, triethyl-, tributyl- or dimethyl-propylamine, or a mono-, di- or trihydroxy lower alkylamine, for example mono-, di- or triethanolamine. Corresponding internal salts may also be formed.

Those skilled in the art of organic and/or medicinal chemistry will appreciate that many organic compounds can form complexes with solvents in which they are reacted or from which they are precipitated or crystallised. These complexes are known as “solvates”. For example, a complex with water is known as a “hydrate”. Solvates, such as hydrates, exist when the drug substance incorporates solvent, such as water, in the crystal lattice in either stoichiometric or non-stoichiometric amounts. Drug substances are routinely screened for the existence of solvates such as hydrates since these may be encountered at any stage. Accordingly, it will be understood that the compounds useful for the present invention may be present in the form of solvates, such as hydrates. Solvated forms of the compounds which are suitable for use in the invention are those wherein the associated solvent is pharmaceutically acceptable. For example, a hydrate is an example of a pharmaceutically acceptable solvate.

The compounds useful for the present invention may be present in amorphous form or crystalline form. Many compounds exist in multiple polymorphic forms, and the use of the compounds in all such forms is encompassed by the present disclosure. Small molecules useful for the present disclosure can be identified using standard procedures such as screening a library of candidate compounds for binding to TNFR2, and then determining if any of the compounds which bind also specifically increase TNFR2-mediated signaling activity. In some embodiments, screening for a compound for use in the invention comprises assessing whether the compound activates TNFR2 signaling activity in cells, for example, by measuring cell proliferation or expression of downstream genes. Small molecules useful for the present invention can also be identified using procedures for in silico screening, which can include screening of known library compounds, to identify candidates which activate TNFR2 signaling activity.

### TNFR2-Specific Antagonists

Examples of types of TNFR2-specific antagonists useful for inhibiting effector T cell activation include, but are not limited to, a small molecule, a polynucleotide, a polypeptide, or a peptide.

### Polypeptides Comprising Antigen Binding Sites

In some embodiments the TNFR2-specific antagonist is a polypeptide, which may inhibit TNFR2 signaling activity by at least one of a number of different mechanisms, e.g., specifically binding to TNFR2 thereby reducing interaction of the receptor with TNFα.

In some embodiments, a TNFR2-specific antagonist is a polypeptide comprising an antigen binding site such as an antibody or a fragment thereof. In some embodiments, the TNFR2-specific antagonist is an antibody or a fragment thereof. Suitable types of TNFR2-specific antagonist antibodies and their methods of production are described above in relation to TNFR2-specific agonists.

Exemplary antibodies that can act as TNFR2-specific antagonists include mAb226 (R&D Systems, Inc., Minneapolis, Minn.), “anti-TNFR2 Ab” (Catalog #HM1374, Hypromatrix, Worcester, Mass.), M861 (Amgen Inc.), TR75-54.7 (Nie et al., 2018), and antibodies described in Torrey et al. (2017) and Okubo et al. (2013 and 2016). In some embodiments, the TNFR2-specific antagonist is mAb226, anti-TNFR2 Ab, TR75-54.7, M861, or a fragment of anyone thereof. In some embodiments, the TNFR2-specific antagonist is an antibody described in in Torrey et al. (2017) or Okubo et al. (2013 and 2016) or a fragment of anyone thereof. In some embodiments, the TNFR2-specific antagonist comprises the CDRs of mAb226, anti-TNFR2 Ab, TR75-54.7, or M861. In some embodiments, the TNFR2-specific antagonist comprises the CDRs of an antibody described in in Torrey et al. (2017) or Okubo et al. (2013 and 2016).

### Small Molecules

In some embodiments, the TNFR2-specific antagonist is a small molecule. In some embodiments, the small molecule binds to TNFR2 and decreases its signaling activity. For example, upon binding of the small molecule to TNFR2 it may competitively inhibit binding of TNFα. Suitable small molecule TNFR2-specific agonists for use in the invention can be identified using screening methods that are routine in the art.

Small molecules useful for the present disclosure can be identified using standard procedures such as screening a library of candidate compounds for binding to TNFR2, and then determining if any of the compounds which bind also specifically inhibit TNFR2-mediated signaling activity. In some embodiments, screening for a compound for use in the invention comprises assessing whether the compound inhibits TNFR2 signaling activity in cells, for example, by measuring cell proliferation or expression of downstream genes. Or by measuring the level of expression or phosphorylation of Etk, as described herein. Small molecules useful for the present invention can also be identified using procedures for in silico screening, which can include screening of known library compounds, to identify candidates which inhibit TNFR2 signaling activity.

Suitable salts, solvents, and forms of small molecule TNFR2-specific antagonists are described above in relation to TNFR2-specific agonists.

### RNA Interference Polynucleotides

In some embodiments the TNFR2-specific antagonist is a polynucleotide, which may inhibit TNFR2 mediated signaling activity by at least one of a number of different mechanisms.

In some embodiments the polynucleotide TNFR2-specific antagonist acts by reducing expression of TNFR2 by targeting its mRNA. For example, the polynucleotide may reduce expression of TNFR2 by RNA interference.

The terms “RNA interference”, “RNAi” or “gene silencing” refer generally to a process in which a double-stranded RNA molecule reduces the expression of a nucleic acid sequence with which the double-stranded RNA molecule shares substantial or total homology. However, it has been shown that RNA interference can also be achieved using non-RNA double stranded molecules (see, for example, US 20070004667).

In some embodiments, a TNFR2-specific antagonist comprises nucleic acid molecules comprising and/or encoding double-stranded regions for RNA interference against TNFR2 mRNA. The nucleic acid molecules are typically RNA, but may comprise chemically-modified nucleotides and non-nucleotides.

The double-stranded regions should be at least 19 contiguous nucleotides, for example about 19 to 23 nucleotides, or may be longer, for example 30 or 50 nucleotides, or 100 nucleotides or more. The full-length sequence corresponding to the entire gene transcript may be used. Preferably, they are about 19 to about 23 nucleotides in length.

The degree of identity of a double-stranded region of a nucleic acid molecule to the targeted transcript should be at least 90% and more preferably 95-100%. The nucleic acid molecule may of course comprise unrelated sequences which may function to stabilize the molecule.

The term “short interfering RNA” or “siRNA” as used herein refers to a nucleic acid molecule which comprises ribonucleotides capable of inhibiting or down regulating gene expression, for example by mediating RNAi in a sequence-specific manner, wherein the double stranded portion is less than 50 nucleotides in length, preferably about 19 to about 23 nucleotides in length. For example, the siRNA can be a nucleic acid molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary.

As used herein, the term siRNA is meant to be equivalent to other terms used to describe nucleic acid molecules that are capable of mediating sequence specific RNAi, for example micro-RNA (miRNA), short hairpin RNA (shRNA), short interfering oligonucleotide, short interfering nucleic acid (siNA), short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siRNA molecules can be used to epigenetically silence genes at both the post-transcriptional level or the pre-transcriptional level. In a non-limiting example, epigenetic regulation of gene expression by siRNA molecules can result from siRNA mediated modification of chromatin structure to alter gene expression.

By “shRNA” or “short-hairpin RNA” is meant an RNA molecule where less than about 50 nucleotides, preferably about 19 to about 23 nucleotides, is base paired with a complementary sequence located on the same RNA molecule, and where said sequence and complementary sequence are separated by an unpaired region of at least about 4 to about 15 nucleotides which forms a single-stranded loop above the stem structure created by the two regions of base complementarity.

Included shRNAs are dual or bi-finger and multi-finger hairpin dsRNAs, in which the RNA molecule comprises two or more of such stem-loop structures separated by single-stranded spacer regions.

Once designed, the nucleic acid molecules comprising a double-stranded region can be generated by any method known in the art, for example, by in vitro transcription, recombinantly, or by synthetic means.

Modifications or analogs of nucleotides can be introduced to improve the properties of the nucleic acid molecules. Improved properties include increased nuclease resistance and/or increased ability to permeate cell membranes. Accordingly, the terms “nucleic acid molecule” and “double-stranded RNA molecule” includes synthetically modified bases such as, but not limited to, inosine, xanthine, hypoxanthine, 2-aminoadenine, 6-methyl-, 2-propyl- and other alkyl-adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

Chemically modified siRNAs particularly suited for in vivo delivery are described in the art in, e.g., WO2014201306, WO2007051303.

### Polynucleotides Encoding Peptides or Polypeptides

In some embodiments, a polynucleotide-based TNFR2-specific antagonist encodes a polypeptide, so that delivery of the polynucleotide to cells results in expression of an encoded peptide or polypeptide TNFR2-specific antagonist. For example, the polynucleotide-based TNFR2-specific antagonist may encode any one or more of the TNFR2-specific antagonist polypeptides or peptides described above.

In some embodiments, the polynucleotide encodes a dominant negative suppressor of TNFR2 mediated signaling activity.

In some embodiments, the polynucleotide TNFR2-specific antagonist encodes a programmable nuclease which inhibits TNFR2 mediated signaling activity by inactivating or reducing expression of the genes encoding TNFR2. As used herein, the term “programmable nuclease” relates to nucleases that are “targeted” (“programmed”) to recognize and edit a pre-determined genomic location. In some embodiments the encoded polypeptide is a programmable nuclease “targeted” or “programmed” to introduce a genetic modification into the TNFR2 encoding gene or regulatory region thereof. In some embodiments, the genetic modification is a deletion or substitution in the TNFR2 encoding gene or in a regulatory region thereof.

In some embodiments, the programmable nuclease may be programmed to recognize a genomic location by a combination of DNA-binding zinc-finger protein (ZFP) domains. ZFPs recognize a specific 3-bp in a DNA sequence, a combination of ZFPs can be used to recognize a specific a specific genomic location. In some embodiments, the programmable nuclease may be programmed to recognize a genomic location by transcription activator-like effectors (TALEs) DNA binding domains. In an alternate embodiment, the programmable nuclease may be programmed to recognize a genomic location by one or more RNA sequences. In an alternate embodiment, the programmable nuclease may be programmed by one or more DNA sequences. In an alternate embodiment, the programmable nuclease may be programmed by one or more hybrid DNA/RNA sequences. In an alternate embodiment, the programmable nuclease may be programmed by one or more of an RNA sequence, a DNA sequences and a hybrid DNA/RNA sequence.

Programmable nucleases that can be used in accordance with the present disclosure include, but are not limited to, RNA-guided engineered nuclease (RGEN) derived from the bacterial clustered regularly interspaced short palindromic repeat (CRISPR)-cas (CRISPR-associated) system, zinc-finger nuclease (ZFN), transcription activator-like nuclease (TALEN), and argonautes.

In some embodiments, the nuclease is a RNA-guided engineered nuclease (RGEN). In some embodiments the RGEN is from an archaeal genome or is a recombinant version thereof. In some embodiments the RGEN is from a bacterial genome or is a recombinant version thereof. In some embodiments the RGEN is from a Type I (CRISPR)-cas (CRISPR-associated) system. In some embodiments the RGEN is from a Type II (CRISPR)-cas (CRISPR-associated) system. In some embodiments the RGEN is from a Type III (CRISPR)-cas (CRISPR-associated) system. In some embodiments the nuclease is a class I RGEN. In some embodiments the nuclease is a class II RGEN. In some embodiments the RGEN is a multi-component enzyme. In some embodiments the RGEN is a single component enzyme. In some embodiments the RGEN is CAS3. In some embodiments, the RGEN is CAS 10. In some embodiments the RGEN is CAS9. In some embodiments, the RGEN is Cpf1 (Zetsche et al., 2015). In some embodiments, the RGEN is targeted by a single RNA or DNA. In some embodiments, the RGEN is targeted by more than one RNA and/or DNA. In some embodiments, the programmable nuclease may be a DNA programmed argonaute (WO 14/189628).

In some embodiments, the polynucleotide TNFR2-specific antagonist is provided in an expression vector to be delivered to cells (e.g., neurons) using any of a number of routine targeting methods known in the art.

As used herein, an “expression vector” is a DNA or RNA vector that is capable of effecting expression of one or more polynucleotides in a host cell (e.g., a neuron). The vector is typically a plasmid or recombinant virus. Any suitable expression vector can be used, examples of which include, but are not limited to, a plasmid or viral vector. In some embodiments, the viral vector is a retrovirus, a lentivirus, an adenovirus, a herpes virus, or an adeno-associated viral vector.

Such vectors will include one or more promoters for expressing the polynucleotide such as a dsRNA for gene silencing. Suitable promoters include include, but are not limited to, the retroviral LTR; the SV40 promoter; and the human cytomegalovirus (CMV) promoter. Cellular promoters such as eukaryotic cellular promoters including, but not limited to, the histone, RNA polymerase III (in the case of snRNA or miRNA expression), and β-actin promoters, can also be used. In some embodiments the promoter is an effector T cell specific promoter. The selection of a suitable promoter will be apparent to those skilled in the art from the teachings contained herein.

### Methods of Treatment

The methods described herein can be used to treat subjects which have a disease which would benefit from an increase in effector T cell activation or a disease which is mediated by over-activation of effector T cells.

Diseases which Would Benefit from an Increase in Effector T Cell Activation

A disease which would benefit from an increase in effector T cell activation is any disease for which increased T cell activation could relieve to some extent or prevent worsening of one or more of the symptoms of the disease or condition being treated. The result can be reduction or a prevention of progression of the signs, symptoms, or causes of a disease, or any other desired alteration of a biological system. Such diseases include, for example, cancer, an infection, or an immunodeficiency.

Cancers that may be treated using the methods of the invention include acute lymphoblastic leukemia, acute lymphoblastic leukemia, acute myeloid leukemia, adrenocortical carcinoma; aids-related lymphoma, aids-related malignancies, anal cancer, astrocytoma, glioblastoma, neuroblastoma, bile duct cancer, bladder cancer, bile duct cancer, bone cancer, osteosarcoma/malignant fibrous histiocytoma, brain stem glioma, visual pathway and hypothalamic glioma, breast cancer, prostate cancer, bronchial adenomas/carcinoids, chronic lymphocytic leukemia, chronic myelogenous leukemia, chronic myeloproliferative disorders, clear cell sarcoma of tendon sheaths, colon cancer, colorectal cancer, cutaneous T cell lymphoma, TNF-secreting T cell lymphoma, endometrial cancer, epithelial cancer, esophageal cancer, Ewing's family of tumours, extracranial germ cell tumour, extragonadal germ cell tumour, parathyroid cancer, extrahepatic bile duct cancer, eye cancer, intraocular melanoma, retinoblastoma, rhabdomyosarcoma, gallbladder cancer, gastric (stomach) cancer, savilary gland cancer, blood cancer, hairy cell leukemia, head and neck cancer, pancreatic cancer, hepatocellular (liver) cancer, hodgkin's lymphoma, hypopharyngeal cancer, kaposi's sarcoma, kidney cancer, laryngeal cancer, merkel cell carcinoma, non-hodgkin's lymphoma, osteosarcoma, ovarian cancer, pituitary cancer, plasma cell neoplasm/multiple myeloma, pleuropulmonary blastoma, sarcoma, skin cancer (non-melanoma), melanoma, small cell lung cancer, small intestine cancer, soft tissue sarcoma, squamous neck cancer, testicular cancer, thyroid cancer, throat cancer, thymoma, thymic carcinoma, urethral cancer, uterine sarcoma, vaginal cancer, and wilms tumour. The cancer can also be solid tumours including malignancies (e.g., sarcomas, adenocarcinomas, and carcinomas) of the various organ systems, such as those of brain, lung, breast, lymphoid, gastrointestinal (e.g., colon), and genitourinary (e.g., renal, urothelial, or testicular tumours) tracts, pharynx, prostate, and ovary. exemplary adenocarcinomas include colorectal cancers, renal-cell carcinoma, liver cancer, non-small cell carcinoma of the lung, and cancer of the small intestine.

Infections that may be treated using the methods of the invention include viral infections such as infections with a member of the Flaviviridae family (e.g., a member of the Flavivirus, Pestivirus, and Hepacivirus genera), which includes the hepatitis C virus, Yellow fever virus; Tick-borne viruses, such as the Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofj in virus, Louping ill virus and the Negishi virus; seabird tick-borne viruses, such as the Meaban virus, Saumarez Reef virus, and the Tyuleniy virus; mosquito-borne viruses, such as the Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus; and viruses with no known arthropod vector, such as the Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, and the Cell fusing agent virus; a member of the Arenaviridae family, which includes the Ippy virus, Lassa virus (e.g., the Josiah, LP, or GA391 strain), lymphocytic choriomeningitis virus (LCMV), Mobala virus, Mopeia virus, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Whitewater Arroyo virus, Chapare virus, and Lujo virus; a member of the Bunyaviridae family (e.g., a member of the Hantavirus, Nairovirus, Orthobunyavirus, and Phlebovirus genera), which includes the Hantaan virus, Sin Nombre virus, Dugbe virus, Bunyamwera virus, Rift Valley fever virus, La Crosse virus, California encephalitis virus, and Crimean-Congo hemorrhagic fever (CCHF) virus; a member of the Filoviridae family, which includes the Ebola virus (e.g., the Zaire, Sudan, Ivory Coast, Reston, and Uganda strains) and the Marburg virus (e.g., the Angola, Ci67, Musoke, Popp, Ravn and Lake Victoria strains); a member of the Togaviridae family (e.g., a member of the Alphavirus genus), which includes the Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, and the chikungunya virus; a member of the Poxviridae family (e.g., a member of the Orthopoxvirus genus), which includes the smallpox virus, monkeypox virus, and vaccinia virus; a member of the Herpesviridae family, which includes the herpes simplex virus (HSV; types 1, 2, and 6), human herpes virus (e.g., types 7 and 8), cytomegalovirus (CMV), Epstein-Barr virus (EBV), Varicella-Zoster virus, and Kaposi's sarcoma associated-herpesvirus (KSHV); a member of the Orthomyxoviridae family, which includes the influenza virus (A, B, and C), such as the H5N1 avian influenza virus or H1N1 swine flu; a member of the Coronaviridae family, which includes the severe acute respiratory syndrome (SARS) virus; a member of the Rhabdoviridae family, which includes the rabies virus and vesicular stomatitis virus (VSV); a member of the Paramyxoviridae family, which includes the human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, and mumps virus; a member of the Picornaviridae family, which includes the poliovirus, human enterovirus (A, B, C, and D), hepatitis A virus, and the coxsackievirus; a member of the Hepadnaviridae family, which includes the hepatitis B virus; a member of the Papillamoviridae family, which includes the human papilloma virus; a member of the Parvoviridae family, which includes the adeno-associated virus; a member of the Astroviridae family, which includes the astrovirus; a member of the Polyomaviridae family, which includes the JC virus, BK virus, and SV40 virus; a member of the Calciviridae family, which includes the Norwalk virus; a member of the Reoviridae family, which includes the rotavirus; and a member of the Retroviridae family, which includes the human immunodeficiency virus (HIV; e.g., types 1 and 2), and human T-lymphotropic virus Types I and II (HTLV-1 and HTLV-2, respectively)).

The methods of the invention can also be used for treating bacterial infections. Examples of bacterial infections that may be treated include, but are not limited to, those caused by bacteria within the genera Salmonella, Streptococcus, Bacillus, Listeria, Corynebacterium, Nocardia, Neisseria, Actinobacter, Moraxella, Enterobacteriacece, Pseudomonas, Escherichia, Klebsiella, Serratia, Enterobacter, Proteus, Salmonella, Shigella, Yersinia, Haemophilus, Bordatella, Legionella, Pasteurella, Francisella, Brucella, Bartonella, Clostridium, Vibrio, Campylobacter, and Staphylococcus.

The methods of the invention can also be used for treating parasitic infections caused by a protozoan parasite (e.g., an intestinal protozoa, a tissue protozoa, or a blood protozoa) or a helminthic parasite (e.g., a nematode, a helminth, an adenophorea, a secementea, a trematode, a fluke (blood flukes, liver flukes, intestinal flukes, and lung flukes), or a cestode). Exemplary protozoan parasites include Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Plasmodium vivax, Plasmodium ovale, Plasmodium malariae, Plasmodium falciparum, Trichomonas vaginalis, and Histomonas meleagridis. Exemplary helminthic parasites include Richuris trichiura, Ascaris lumbricoides, Enterobius vermicularis, Ancylostoma duodenale, Necator americanus, Strongyloides stercoralis, Wuchereria bancrofti, and Dracunculus medinensis, Schistosoma mansoni, Schistosoma haematobium, Schistosoma japonicum, Fasciola hepatica, Fasciola gigantica, Heterophyes heterophyes, and Paragonimus westermani, Taenia solium, Taenia saginata, Hymenolepis nana, and Echinococcus granulosus.

The methods of the invention can also be used for treating fungal infections. Examples of fungal infections that may be treated include, but are not limited to, those caused by, e.g., Aspergillus, Candida, Malassezia, Trichosporon, Fusarium, Acremonium, Rhizopus, Mucor, Pneumocystis, and Absidia.

The methods of the invention can also be used for treating immunodeficiency disorders. Examples of immunodeficiency disorders that may be treated, include, for example, Agammaglobulinemia: X-Linked and Autosomal Recessive, Ataxia Telangiectasia, Chronic Granulomatous Disease and Other Phagocytic Cell Disorders, Common Variable Immune Deficiency, Complement Deficiencies, DiGeorge Syndrome, Hemophagocytic Lymphohistiocytosis (HLH), Hyper IgE Syndrome, Hyper IgM Syndromes, IgG Subclass Deficiency, Innate Immune Defects, NEMO Deficiency Syndrome, Selective IgA Deficiency, Selective IgM Deficiency, Severe Combined Immune Deficiency and Combined Immune Deficiency, Specific Antibody Deficiency, Transient Hypogammaglobulinemia of Infancy, WHIM Syndrome (Warts, Hypogammaglobulinemia, Infections, and Myelokathexis), and Wiskott-Aldrich Syndrome.

### Diseases Mediated by Over-Activation of Effector T Cells

Diseases mediated by over-activation of effector T cells include, for example, autoimmune diseases and/or inflammatory diseases.

Autoimmune and inflammatory diseases that may be treated using the methods of the invention include Type I diabetes, Type II Diabetes, Alopecia Areata, Ankylosing Spondylitis, Antiphospholipid Syndrome, Asthma, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Essential Mixed Cryoglobulinemia, Fibromyalgia-Fibromyositis, Graft vs Host Disease (GVHD), Graves' Disease, Guillain-Barre, Hashimoto's Thyroiditis, Hidradenitis Suppurativa, Hypothyroidism, Idiopathic Pulmonary Fibrosis, Idiopathic Thrombocytopenia Purpura (ITP), Inflammatory Bowel Disease, IgA Nephropathy, Juvenile Arthritis, Lichen Planus, Lupus, Meniere's Disease, Mixed Connective Tissue Disease, Multiple Sclerosis, Myasthenia Gravis, Osteomyelitis, Pemphigus Vulgaris, Pernicious Anemia, Polyarteritis Nodosa, Polyarthritis, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reactive Arthritis, Reiter's Syndrome, Rheumatic Fever, Rheumatoid Arthritis, Sarcoidosis, Scleroderma, Sjogren's Syndrome, Stiff-Man Syndrome, Takayasu Arteritis, Temporal Arteritis/Giant Cell Arteritis, Toxic Shock Syndrome, Transplant Rejection, Ulcerative Colitis, Uveitis, Vasculitis, Vitiligo, and Wegener's Granulomatosis. Additional autoimmune diseases that can be treated using the methods of the invention are disclosed in U.S. Pat. No. 8,173,129.

### Administration of Agonists and Antagonists

In some embodiments, methods of treatment described herein include administration of a pharmaceutical composition containing at least one TNFR2-specific agonist or antagonist, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In some embodiments, a TNFR2-specific agonist or antagonist, is administered to treat, prevent, or at least partially arrest the symptoms of a subject already suffering from and/or diagnosed as having either a disease which would benefit from an increase in effector T cell activation or a disease mediated by over-activation of effector T cells. Amounts effective for this use will depend on the severity and course of the disease, previous therapy, the subject's health status, weight, and response to the treatment. It is considered well within the skill of the art for one to determine such therapeutically effective amounts by routine experimentation (including, but not limited to, a dose escalation clinical trial).

In a case where a subject's status does improve, upon reliable medical advice, the administration of a TNFR2-specific agonist or antagonist may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, or 60 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

The amount of a given TNFR2-specific agonist or antagonist that will be suitable as a therapeutically effective dose will vary depending upon factors such as the type and potency of the TNFR2-specific agonist or antagonist to be administered, the severity/stage of the subject's disease, the characteristics (e.g., weight) of the subject in need of treatment, and prior or concurrent treatments, but can nevertheless be routinely determined in a manner known in the art according to the particular circumstances surrounding the case, including, e.g., the specific agent being administered, the route of administration, the condition being treated or prevented, and the subject or host being treated. In general, however, doses employed for adult human treatment will typically be in the range of 0.02-5000 mg per day, or from about 1-1500 mg per day. The desired dose may conveniently be presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages may be altered depending on a number of variables, not limited to the activity of the TNFR2-specific agonist or antagonist to be used, the type and severity of disease to be treated or prevented, the mode of administration, and the judgment of the practitioner.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. TNFR2-specific agonists or antagonists exhibiting high therapeutic indices are preferred. The data obtained from cell culture assays and animal studies can be used in formulating a range of dosage for use in human and non-human subjects. The dosage of such compounds lies preferably within a range of circulating concentrations that include the ED₅₀ with minimal toxicity. The dosage may vary within this range depending upon the dosage form employed and the route of administration utilized.

### Combination Treatments

TNFR2-specific agonists or antagonists can also be used in combination with other agents of therapeutic value for increasing or inhibiting activation of effector T cells. In general, other agents do not necessarily have to be administered in the same pharmaceutical composition, and may, because of different physical and chemical characteristics, preferably be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

A TNFR2-specific agonist or antagonist and an additional therapeutic agent may be administered concurrently (e.g., simultaneously, essentially simultaneously or within the same treatment protocol) or sequentially, depending upon the nature and phase of the infection, the condition of the subject, and the actual choice of therapeutic agents used. The determination of the order of administration, and the number of repetitions of administration of each therapeutic agent during a treatment protocol, is well within the knowledge of the skilled physician after evaluation of the disease being treated or prevented and the condition of the subject.

It is known to those of skill in the art that therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the subject.

For combination therapies, dosages of co-administered therapeutic agents will of course vary depending on the type of co-agents employed, on the specific TNFR2-specific agonist or antagonist, and disease to be treated or prevented.

It is understood that the dosage regimen to treat, prevent, or ameliorate the condition(s) for which relief is sought, can be modified in accordance with a variety of factors. These factors include the condition from which the subject suffers, as well as the age, weight, sex, diet, and general medical condition of the subject. Thus, the dosage regimen actually employed can vary widely and therefore can deviate from the dosage regimens set forth herein.

The TNFR2-specific agonist or antagonist and additional therapeutic agent which make up a combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of various physiological parameters may also be evaluated to determine the optimal dose interval.

In addition, administration or co-administration of a TNFR2-specific agonist or antagonist may be used in combination with procedures that may provide additional or synergistic benefit to the subject. By way of example only, subjects may undergo genetic testing to identify genetic variation in their genome so as to optimize treatment parameters, e.g., the type of TNFR2-specific agonist or antagonist to be administered, dosing regimen, and co-administration with additional therapeutic agents.

Initial administration can be via any route practical, such as, for example, an intravenous injection, a bolus injection, infusion over 5 minutes to about 5 hours, a pill, a capsule, inhaler, injection, transdermal patch, buccal delivery, and the like, or combination thereof. A compound should be administered as soon as is practicable after the onset of a disease or condition is detected or suspected, and for a length of time necessary for the treatment of the disease being treated.

In some embodiments, the TNFR2-specific agonist or antagonist is administered in combination with an immunotherapy. An “immunotherapy” is any form of therapy which activates or suppresses the immune system. In some embodiments, the immunotherapy is a polypeptide. In some embodiments, the polypeptide is an antibody or fragment thereof. Antibody-based immunotherapies, such as monoclonal antibodies, antibody-fusion proteins, and antibody drug conjugates (ADCs) are used to treat a wide variety of diseases, including many types of cancer. Such therapies may depend on recognition of cell surface molecules that are differentially expressed on cells for which elimination is desired (e.g., target cells such as cancer cells) relative to normal cells (e.g., non-cancer cells). Binding of an antibody-based immunotherapy to a cancer cell can lead to cancer cell death via various mechanisms, e.g., antibody-dependent cell-mediated cytotoxicity (ADCC), complement-dependent cytotoxicity (CDC), or direct cytotoxic activity of the payload from an antibody-drug conjugate (ADC). Methods of producing and optimising antibodies are described herein. Suitable antibodies include Adalimumab, Ado-Trastuzumab emtansine, Alemtuzumab, Basiliximab, Bevacizumab, Belimumab, Brentuximab, Canakinumab, Cetuximab, Daclizumab, Denosumab, Dinutuximab, Eculizumab, Efalizumab, Epratuzumab, Gemtuzumab, Golimumab, Infliximab, Ipilimumab, Labetuzumab, Natalizumab, Obinutuzumab, Ofatumumab, Omalizumab, Palivizumab, Panitumumab, Pertuzumab, Ramucirumab, Ritutimab, Tocilizumab, Tratuzumab, Ustekinumab, and Vedolizumab.

In some embodiments, the immunotherapy is a cell-based therapy such as a T cell-based immunotherapy. Without wishing to be bound by theory, it is believed that T cell-based immunotherapies may be improved by co-administration of a TNFR2-specific agonist by increasing activation of the T cells being administered.

In some embodiments, the T cell-based immunotherapy is a CAR-T cell therapy. A “chimeric Antigen Receptor” or alternatively a “CAR”, refers to a recombinant polypeptide construct comprising at least an extracellular antigen binding domain, a transmembrane domain and a cytoplasmic signaling domain (also referred to as an intracellular signaling domain) comprising a functional signaling domain derived from a stimulatory molecule and/or costimulatory molecule. An exemplary antigen binding domain is one which binds to a tumour antigen or a viral antigen expressed on a cell surface. Suitable CAR-T cells and methods of production are described in Miliotou and Papadopoulou (2018), June et al. (2018), and June and Sadelain (2018).

TNFR2-specific agonists described herein can be used to enhance the T cell immune response elicited by vaccines by increasing activation of effector T cells. Thus, in some embodiments, the TNFR2-specific agonist is administered as a vaccine adjuvant. In some embodiments, the vaccine is a T cell vaccine. A T cell vaccine is a vaccine designed to induce protective T cells in order to induce effective cellular immunity. They are also referred to as “cell-mediated immune” (CMI) vaccines or T cell-inducing vaccines. Such T cell vaccines are described in Gilbert (2012), for example.

The present inventors have surprisingly found that TNFR2-specific agonists and SMAC mimetics act synergistically to increase effector T cell activation when administered in combination. Thus, in some embodiments, the TNFR2-specific agonist is administered in combination with a SMAC mimetic. SMAC mimetics are compounds that mimic the activity of second mitochondria-derived activator of caspase (SMAC). SMAC is a pro-apoptotic mitochondrial protein that is an endogenous inhibitor of IAPs. SMAC mimetics have been shown to stimulate programmed cell death and thus have become a focus in the development of cancer therapeutics. Examples of SMAC mimetics include birinapant, Debio 1143, CUDC-427, LCL161, AEG40826, ASTX-660, LBW-242, AZD5582, AEG40730, APG-1387, CompA, GDC-0145, GDC-0152, CS3, BV6, MV1, SM-164, AT406, ML101, or embelin. Other SMAC mimetics are described in U.S. Pat. Nos. 7,517,906, 7,419,975, 7,589,118, 7,932,382, 7,345,081, 7,244,851, 7,674,787, 7,772,177, 7,989,441, 8,716,236, US20100324083, US20100056467, US20090069294, US20110065726, US20110206690, WO2013127729, WO2014009495, WO2011098904, WO2013127729, WO2014090709, WO2014085489, WO2014031487, WO2013103703, WO2014055461, WO2014025759, and WO2014011712.

In some embodiments, the TNFR2-specific agonist is administered in combination with a T cell receptor (TCR) agonist. As used herein, “T cell receptor agonists” are compounds that are capable of inducing TCR signaling required for T cell activation (i.e., TCR crosslinking). Such TCR agonists include anti-CD3 antibodies, for example. In some embodiments, the TCR agonist is anti-CD3 antibody, phytohaemaglutinin (PHA), phorbol myristate acetate (PMA), or ionomycin. In some embodiments, the T cell receptor agonist is a Tribody. In some embodiments, the tribody is Tb535.

In some embodiments, the TNFR2-specific agonist is administered in combination with a checkpoint inhibitor. Checkpoint inhibitors antagonise immune checkpoint pathways and act to prevent cancer cells escaping immune surveillance by co-opting such pathways. Because many such immune checkpoints are initiated by ligand-receptor interactions, they can be blocked by antibodies against the ligands and/or their receptors (Pardoll, 2012). Although checkpoint inhibitor antibodies against PD1, PD-L1, and CTLA4 are the most clinically advanced, other potential checkpoint antigens are known and may be used as the target of therapeutic antibodies, such as LAG3, B7-H3, B7-H4 and TIM3. Exemplary anti-PD1 antibodies include pembrolizumab (MK-3475, Merck), nivolumab (BMS-936558, Bristol-Myers Squibb), AMP-224 (Merck), and pidilizumab (CT-011, Curetech Ltd.). Anti-PD1 antibodies are commercially available, for example from Abcam® (AB137132), Biolegend® (EH12.2H7, RMP1-14) and Affymetrix Ebioscience (J105, J116, MIH4). Exemplary anti-PD-L1 antibodies include atezolizumab, MDX-1105 (Medarex), MEDI4736 (Medimmune) MPDL3280A (Genentech) and BMS-936559 (Bristol-Myers Squibb). Anti-PD-L1 antibodies are also commercially available, for example from Affymetrix Ebioscience (MIH1). Exemplary anti-CTLA4 antibodies include ipilimumab (Bristol-Myers Squibb) and tremelimumab (Pfizer). Anti-PD1 antibodies are commercially available, for example from Abcam® (AB134090), Sino Biological Inc. (11159-H03H, 11159-H08H), and Thermo Scientific Pierce (PA5-29572, PA5-23967, PA5-26465, MA1-12205, MA1-35914). Ipilimumab has recently received FDA approval for treatment of metastatic melanoma (Wada et al., 2013).

In addition to checkpoint inhibitors other known agents that stimulate an immune response against e.g., tumours and/or pathogens may be used in combination with TNFR2-specific agonists. Other known co-stimulatory pathway modulators that may be used in combination include, but are not limited to, agatolimod, belatacept, blinatumomab, CD40 ligand, anti-B7-1 antibody, anti-B7-2 antibody, anti-B7-H4 antibody, AG4263, eritoran, anti-OX40 antibody, ISF-154, and SGN-70; B7-1, B7-2, ICAM-1, ICAM-2, ICAM-3, CD48, LFA-3, CD30 ligand, CD40 ligand, heat stable antigen, B7h, OX40 ligand, LIGHT, CD70 and CD24.

In some embodiments, the TNFR2 antagonist is administered in combination with a TNFα antagonist. Antagonists of TNFα include monoclonal antibodies such as infliximab (Remicade), adalimumab (Humira), certolizumab (Cimzia), and golimumab (Simponi), as well as TNF receptor fusion proteins such as etanercept (Enbrel). Thalidomide (Immunoprin) and its derivatives lenalidomide (Revlimid) and pomalidomide (Pomalyst, Imnovid) also act as TNFα antagonists.

In some embodiments, the TNFR2 antagonist is administered in combination with an anti-inflammatory agent. In some embodiments, the anti-inflammatory agent is a non-steroidal anti-inflammatory drug (NSAID). In some embodiments, the anti-inflammatory agent is a cytokine inhibitor, for example a molecule that binds to TNFα, IL-6, IL-1, or IFNβ and prevents them from binding to their receptor.

In some embodiments, the TNFR2-specific antagonist is administered in combination with a CD52 antagonist, CD20 antagonist, or IL-17A antagonist. Examples of such antagonists include alemtuzumab, rituximab, ocrelizumab, obinutuzumab, ofatumumab, ibritumomab tiuxetan, tositumomab, ublituximab, secukinumab, ixekizumab, and brodalumab.

### Compositions and Dosage Forms

Compositions comprising TNFR2-specific agonists or antagonists can be formulated for administration to a subject via any conventional means including, but not limited to, oral, parenteral (e.g., intravenous, subcutaneous, or intramuscular), buccal, inhalation, intranasal, rectal or transdermal administration routes.

In some embodiments, the composition comprises at least two agents for increasing activation of effector T cells, wherein one of the agents is a TNFR2-specific agonist described herein. In some embodiments, the composition comprises at least two agents for treating cancer, infection or an immunodeficiency, wherein one of the agents is a TNFR2-specific agonist.

In some embodiments, the composition comprises at least two agents for inhibiting activation of T cells in a subject, wherein one of the agents is a TNFR2-specific antagonist. In some embodiments, the composition comprises at least two agents for treating an autoimmune disease, inflammatory disease or non-TNF secreting T cell lymphoma, wherein one of the agents is a TNFR2-specific antagonist.

The compositions which include a TNFR2-specific agonist or antagonist alone or in combination with one or more other therapeutic agents, can be formulated into any suitable pharmaceutical dosage form, including but not limited to, aqueous oral dispersions, liquids, mists, gels, syrups, elixirs, slurries, suspensions and the like, for oral ingestion by a subject to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Microencapsulated formulations of a TNFR2-specific agonist or antagonist may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

The pharmaceutical solid oral dosage forms including formulations can be further formulated to provide a controlled release of the TNFR2-specific agonist or antagonist. Controlled release refers to the release of one or more active agents from a dosage form in which they are incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms can be formulated as enteric coated delayed release oral dosage forms, i.e., as an oral dosage form of a pharmaceutical composition which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions to achieve delivery to the lower gastrointestinal tract. In some embodiments the polymers are anionic carboxylic polymers.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

In other embodiments, TNFR2-specific agonist or antagonist formulations are delivered using a pulsatile dosage form. A pulsatile dosage form is capable of providing one or more immediate release pulses at predetermined time points after a controlled lag time or at specific sites. Pulsatile dosage forms may be administered using a variety of pulsatile formulations known in the art. For example, such formulations include, but are not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284. In one embodiment, the controlled release dosage form is pulsatile release solid oral dosage form including at least two groups of particles, (i.e. multiparticulate) each containing a formulation. The first group of particles provides a substantially immediate dose of the TNFR2-specific agonist or antagonist upon ingestion. The first group of particles can be either uncoated or include a coating and/or sealant. The second group of particles includes coated particles, which includes from about 2% to about 75%, from about 2.5% to about 70%, or from about 40% to about 70%, by weight of the total dose of the active agents in the formulation, in admixture with one or more binders. The coating includes a pharmaceutically acceptable ingredient in an amount sufficient to provide a delay of from about 2 hours to about 7 hours following ingestion before release of the second dose. Suitable coatings include one or more differentially degradable coatings such as, by way of example only, pH sensitive coatings (enteric coatings) such as acrylic resins either alone or blended with cellulose derivatives, e.g., ethylcellulose, or non-enteric coatings having variable thickness to provide differential release of the formulation.

Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

Liquid formulation dosage forms for oral administration can be aqueous suspensions selected from the group including, but not limited to, pharmaceutically acceptable aqueous oral dispersions, emulsions, solutions, elixirs, gels, and syrups.

The aqueous suspensions and dispersions can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

### Injectable Formulations

Formulations suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. The pharmaceutical composition may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The pharmaceutical compositions may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

### In Vitro Methods

Effector T cells are utilized in a wide variety of in vitro, in vivo, clinical research and therapeutic applications. Examples include studies of immune response, T cell receptor signaling, cytokine release and gene expression profiling. In particular, isolation and subsequent ex vivo engineering of T lymphocytes for subsequent transplantation into clinical patients is used for cancer therapy. The main approaches to this are engineering of T cells to express either chimeric antigen receptors (CAR) or T cell receptors (TCR). In both approaches, effector T cells are isolated from whole blood, activated and expanded ex vivo, and subsequently infused into human subjects.

The present inventors have found that TNFR2 is a potent co-stimulatory receptor in effector T cells, whose signaling promotes cell proliferation. This surprising finding opens up avenues for expanding populations of effector T cells in vitro, for example after being isolated from a subject for adoptive T cell therapy.

In some embodiments, prior to expansion, a source of effector T cells is obtained from a subject. The effector T cells can be obtained from a number of sources, including peripheral blood mononuclear cells, bone marrow, lymph node tissue, spleen tissue, and tumours. In certain embodiments, any number of T cell lines available in the art, may be used. In certain embodiments, T cells can be obtained from a unit of blood collected from a subject using any number of techniques known to the skilled artisan, such as Ficoll separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, a Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, effector T cells are isolated from peripheral blood lymphocytes by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, in one embodiment, effector T cells are isolated by incubation with anti-CD3/anti-CD28-conjugated beads, such as DYNABEADS M-450 CD3/CD28 T, or XCYTE DYNABEADS for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours. In one embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumour infiltrating lymphocytes (TIL) from tumour tissue or from immunocompromised individuals. Further, use of longer incubation times can increase the efficiency of capture of CD8+ T cells, for example.

Enrichment of an effector T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Effector T cells for use in the in vitro expansion methods described herein may also be antigen-specific T cells. For example, tumour-specific T cells can be used. In certain embodiments, antigen-specific T cells can be isolated from a subject of interest, such as a subject afflicted with a cancer or an infectious disease as described herein. In certain embodiments, antigen-specific T cells can be induced by vaccination of a subject with a particular antigen, either alone or in conjunction with an adjuvant or pulsed on dendritic cells. Antigen-specific cells may also be generated in vitro using any number of methods known in the art, for example, as described in U.S. 60/469,122, U.S. Pat. Nos. 6,040,177, and 5,872,642.

As described herein, activation and expansion of effector T cells requires two signals, a first TCR signal and a second co-stimulatory signal. Accordingly, in an embodiment, the first signal for activation of T cells is provided by stimulating the T cell TCR/CD3 complex. For example, an anti-CD3 monoclonal antibody can be used to activate a population of T cells via the TCR/CD3 complex. A number of anti-human CD3 monoclonal antibodies are commercially available, exemplary antibodies are OKT3 and monoclonal antibody G19-4. The first signal for activation can also be delivered to the effector T cell through other mechanisms. For example, a combination that may be used includes a protein kinase C (PKC) activator, such as a phorbol ester (e.g., phorbol myristate acetate), and a calcium ionophore (e.g., ionomycin, which raises cytoplasmic calcium concentrations), or the like. The use of such agents bypasses the TCR/CD3 complex but delivers a stimulatory signal to T cells. Other agents acting as primary signals may include natural and synthetic ligands. A natural ligand may include MHC with or without a peptide presented. Other ligands may include, but are not limited to, a peptide, polypeptide, growth factor, cytokine, chemokine, glycopeptide, soluble receptor, steroid, hormone, mitogen, such as PHA, or other superantigens, peptide-MHC tetramers (Altman et al., 1996) and soluble MHC dimers (Dal Porto et al., 1993).

The second co-stimulatory signal for expanding the effector T cells can be provided by any one or more TNFR2-specific agonists described herein. In some embodiments, the effector T cells are also contacted with a molecule that binds another co-stimulatory molecule, in addition to TNFR2. Other co-stimulatory molecules include CD28, ICOS, 41-BB, and OX40 for example. Accordingly, one of skill in the art will recognise that any agent, including their natural ligands or an antibody or fragment thereof, capable of cross-linking these co-stimulatory molecules can be used in combination with the TNFR2-specific agonists described herein. Exemplary anti-CD28 antibodies or fragments thereof include monoclonal antibody 9.3 (IgG2) (Bristol-Myers Squibb, Princeton, N.J.), monoclonal antibody KOLT-2 (IgG1), 15E8 (IgG1), 248.23.2 (IgM), and EX5.3D10 (IgG2) (ATCC HB11373). Exemplary natural ligands include the B7 family of proteins, such as B7-1 (CD80) and B7-2 (CD86) (Freedman et al., 1987; Freeman et al., 1989).

### Kits

Also provided herein are kits comprising TNFR2-specific agonists or antagonists.

In one embodiment, the kit comprises (a) a container comprising a TNFR2-specific agonist as described herein, optionally in a pharmaceutically acceptable carrier or diluent; and (b) a package insert with instructions for treating a subject who has a disease which would benefit from an increase in effector T cell activation as described herein.

In another embodiment, the kit comprises (a) a container comprising a TNFR2-specific antagonist as described herein, optionally in a pharmaceutically acceptable carrier or diluent; and (b) a package insert with instructions for treating a subject who has a disease mediated by over-activation of effector T cells as described herein.

In accordance with this example of the disclosure, the package insert is on or associated with the container. Suitable containers include, for example, bottles, vials, syringes, etc. The containers may be formed from a variety of materials such as glass or plastic. The container holds or contains a TNFR2-specific agonist or antagonist and may have a sterile access port (for example, the container may be an intravenous solution bag or a vial having a stopper pierceable by a hypodermic injection needle). At least one active agent in the composition is the TNFR2-specific agonist or antagonist. The label or package insert indicates that the composition is used for administration to a subject eligible for treatment, e.g., one having or predisposed to the disease to be treated, with specific guidance regarding dosing amounts and intervals of compound and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

In one embodiment, the kit comprises at least two agents for increasing activation of effector T cells, wherein one of the agents is a TNFR2-specific agonist described herein. In one embodiment, the kit comprises at least two agents for treating cancer, infection or an immunodeficiency, wherein one of the agents is a TNFR2-specific agonist described herein.

In one embodiment, the kit comprises at least two agents for inhibiting activation of T cells in a subject, wherein one of the agents is a TNFR2-specific antagonist described herein. In one embodiment, the kit comprises at least two agents for treating an autoimmune disease, inflammatory disease or non-TNF secreting T cell lymphoma, wherein one of the agents is a TNFR2-specific antagonist described herein.

### EXAMPLES

### Example 1

### Materials and Methods

### Mice

All experiments were performed using C57BL/6 mice bred and maintained under specific pathogen-free conditions in the WEHI animal facilities (Parkville, Victoria, Australia) and used between 8-20 weeks of age. All experiments were performed under the approval of the WEHI Animal Ethics Committee. Tnfr1−/− and Tnfr2−/− mice were maintained at the WEHI.

### T Cell Isolation

T cells were isolated from pooled mouse lymph nodes (inguinal, axillary, brachial, superficial cervical and lumbar) and isolated using MACS negative selection kits for CD4+ or CD8+ T cells (Miltenyi Biotec, Auburn, Calif., USA). T cell purity was confirmed by flow cytometry following isolation. For all experiments, T cell purity was between 82.9-98.2%.

### Cell Culture

T cells were cultured in RPMI 1640 medium (Invitrogen) supplemented with non-essential amino acids, 1 mM Sodium-pyruvate, 10 mM HEPES, 100 U/ml Penicillin, 100 μg/ml Streptomycin (Invitrogen), 50 μM 2β-mercaptoethanol, 2 mM L-glutamine (Sigma) and 10% FCS (Sigma). Cells were incubated in a humidified environment at 37° C. in 5% CO2. For division tracking experiments, T cells were labelled with Cell Trace Violet (Invitrogen) according to the manufacturer's instructions. T cells were cultured in flat bottom tissue culture plates.

The following T cell stimulations were used: TCR was stimulated using 10 μg/ml plate bound anti-CD3 (clone 145-2c11, WEHI antibody facility, Australia) unless a different concentration was specified. Recombinant murine TNF was purchased from R&D Systems (Minneapolis, Minn., USA). Recombinant mIL-2 was purchased from Peprotech. Smac-mimetics were purchased from Sellekchem, MA. The TNFR2 agonist mSTAR was prepared according from HEK293 cells were transiently transfected and cells cultured for 5-7 days, supernatants were purified using anti-Flag affinity chromatography, and the concentrations of the recombinant proteins were determined compared to standard proteins.

### Quantification of Total Cell Numbers.

Triplicate wells were harvested at each time point. To determine absolute cell numbers, the number of live cells per culture was estimated by reference to a known number of Calibrite beads (Becton Dickinson, San Jose, Calif.), added directly to cell culture before collecting. Dead cells were identified via uptake of propidium iodide (SigmaAldrich) added to culture before flow cytometry. Flow cytometry was performed on a BD FACS Canto II or Fortessa.

### Cytokine Bead Array

Supernatant from T cell cultures 48 hours after activation as described above were used to measure cytokine production (IL-2, IL-4, IL-6, IFN-γ, TNF, IL-17A and IL-10) using the Th1/Th2/Th17 BD Cytometric Bead Array (CBA) Kit (BD Bioscience). The CBA assay was performed according to manufacturer's instructions.

### Example 2

### TNFα Stimulates Proliferation of CD4 and CD8 T Cells

To examine the role of TNFα in T cell co-stimulation, naïve CD4 T cells were isolated from C57Bl/6 mice and stimulated with a range of recombinant mouse TNF concentrations in the presence of T cell receptor (TCR) crosslinking via plate bound anti-CD3 stimulation. CD4 T cells were labelled with the cell division tracking dye cell trace violet (CTV) and the response measured daily over 96 hours by quantifying T cell expansion using flow cytometry.

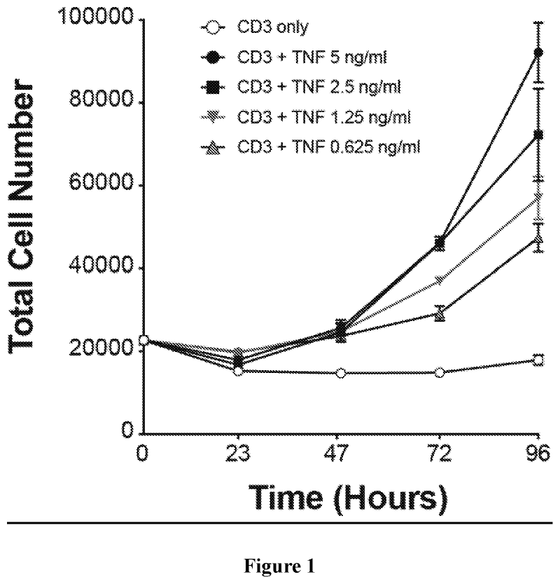

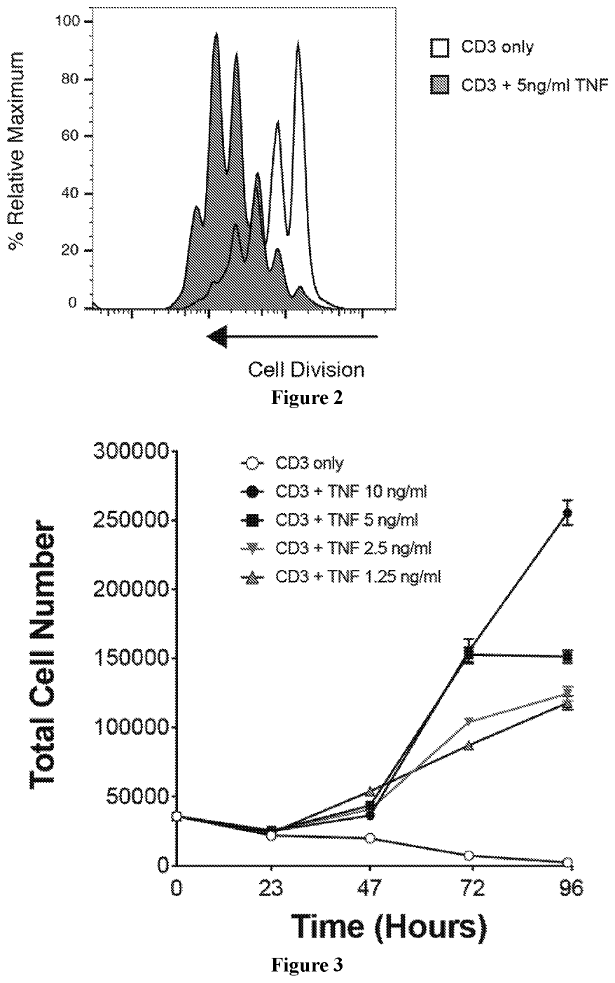

Addition of TNFα significantly enhanced CD4 T cell expansion in a dose dependent manner as determined by total live cell number (FIG. 1) and dilution of cell division dyes (FIG. 2).

To determine if TNF co-stimulation was restricted to CD4 T cells, or could act on all effector cells from the T cell lineage, co-stimulation of naïve CD8 T cells by TNFα was measured. Naïve CTV labelled CD8 T cells were cultured in the presence of plate bound anti-CD3 with various concentrations of recombinant mouse TNFα and the response measured by flow cytometry.

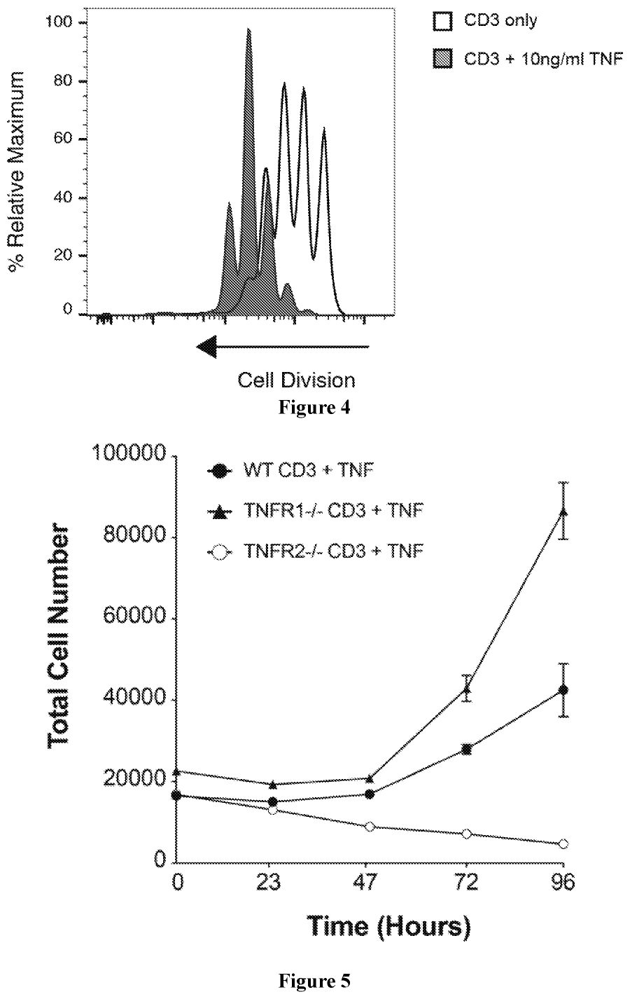

TNF was found to act as a potent co-stimulator of CD8 T cells as measured by expansion of total CD8 T cells numbers (FIG. 3) and dilution of division tracking dyes (FIG. 4).

### Example 3

### TNFα Co-Stimulation of Effector T Cells is Mediated by TNFR2

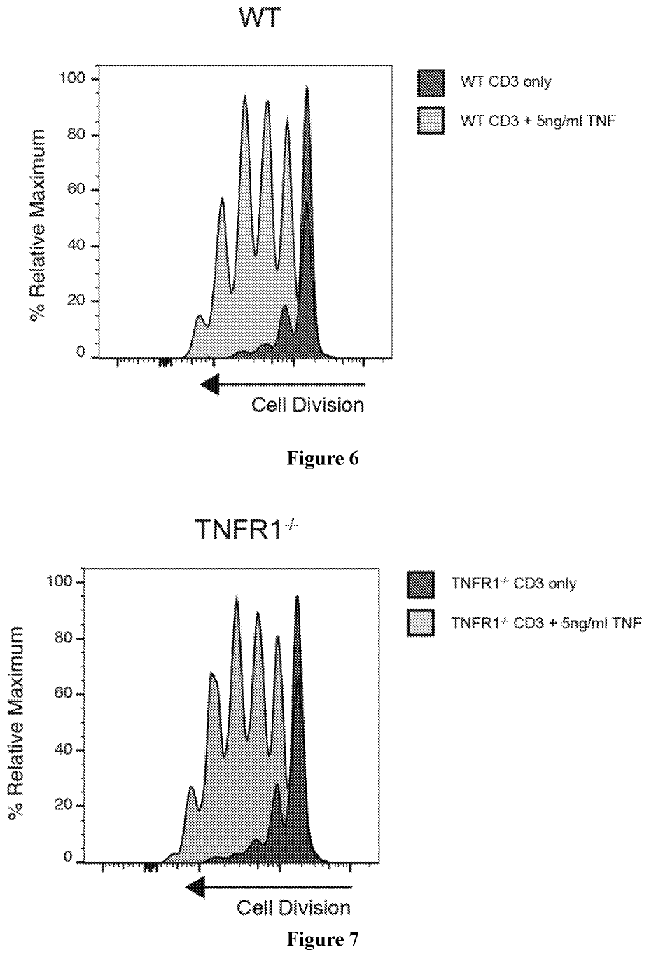

To determine the role of TNF receptors 1 (TNFR1) and 2 (TNFR2) as regulators of TNFα driven T cell co-stimulation, the response of T cells deficient for either TNFR1 or TNFR2 was measured. Naïve CD4 T cells were isolated from WT, tnfr1−/− or tnfr2−/− mice, labelled with CTV, and cultured with recombinant mouse TNFα (5 ng/ml) in the presence of TCR crosslinking (anti-CD3). Expansion of CD4 T cells was measured by total cell numbers (FIG. 5) and dilution of cell tracking dyes (FIGS. 6 to 9) by flow cytometry over 4 days in culture. Surprisingly, the co-stimulation of CD4 T cells by TNF was found to act exclusively through TNFR2.

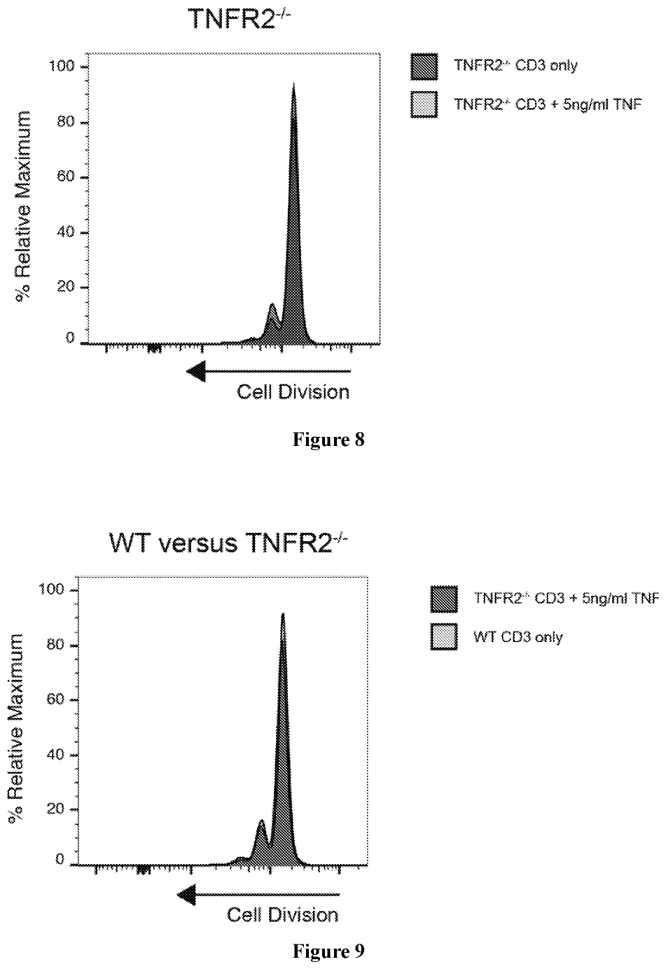

The response of tnfr1−/− CD4 T cells (FIG. 7) was indistinguishable from WT CD4 T cells (FIG. 6) while no co-stimulation was noted in the absence of TNFR2 (FIG. 8). TNFα co-stimulation could be entirely attributed to TNFR2, as tnfr2−/− CD4 T cells co-stimulated with TNFα responded in an identical fashion to WT CD4 T cells stimulated with only anti-CD3 crosslinking (FIG. 9).

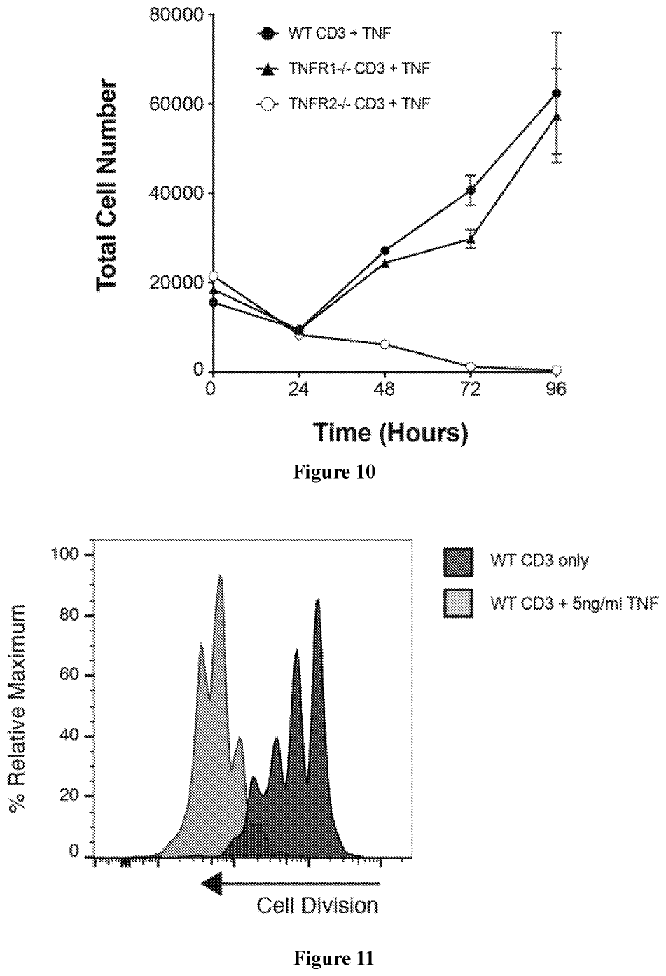

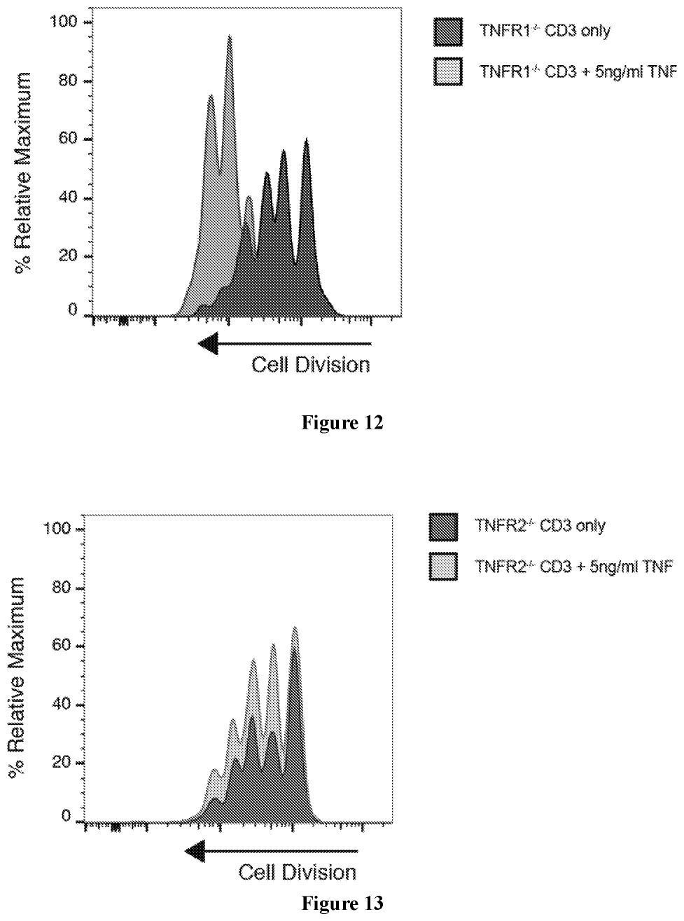

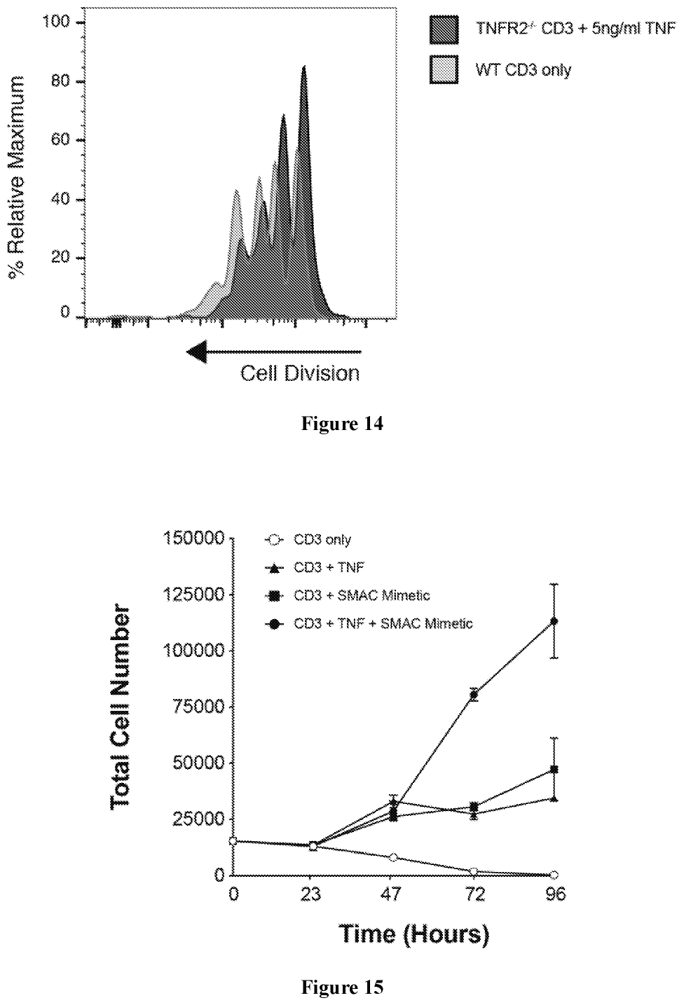

Similar results were noted for naïve CD8 T cells isolated from WT, tnfr1−/− or tnfr2−/− mice (FIGS. 10 to 14). Thus, together these results demonstrated that the co-stimulation of effector T cells by TNFα is mediated exclusively by TNFR2.

### Example 4

### Co-Administration of TNF and SMAC Mimetics

Molecular targets of SMAC mimetics have been suggested to act downstream of TNFR2. Therefore, it was determined whether TNF co-stimulation could synergise with SMAC mimetics to enhance the response of CD8 T cells that had been activated by TCR crosslinking (anti-CD3).

Naïve CD8 T cells were isolated from WT mice and stimulated with combinations of anti-CD3, TNF and SMAC mimetics. The expansion of CD8 T cells was determined by measuring T cell numbers quantified relative to a known number of quantification beads via flow cytometry. CD8 T cells co-stimulated with recombinant mouse TNF (5 ng/ml) yielded a similar response to anti-CD3 stimulated CD8 T cells cultured with SMAC mimetics (FIG. 15). Surprisingly, co-stimulation of anti-CD3 activated CD8 T cells with both TNF and SMAC mimetics synergised to elicit a significantly enhanced T cell response (FIG. 15).

### Example 5

### Administration of a TNFR2-Specific Agonist

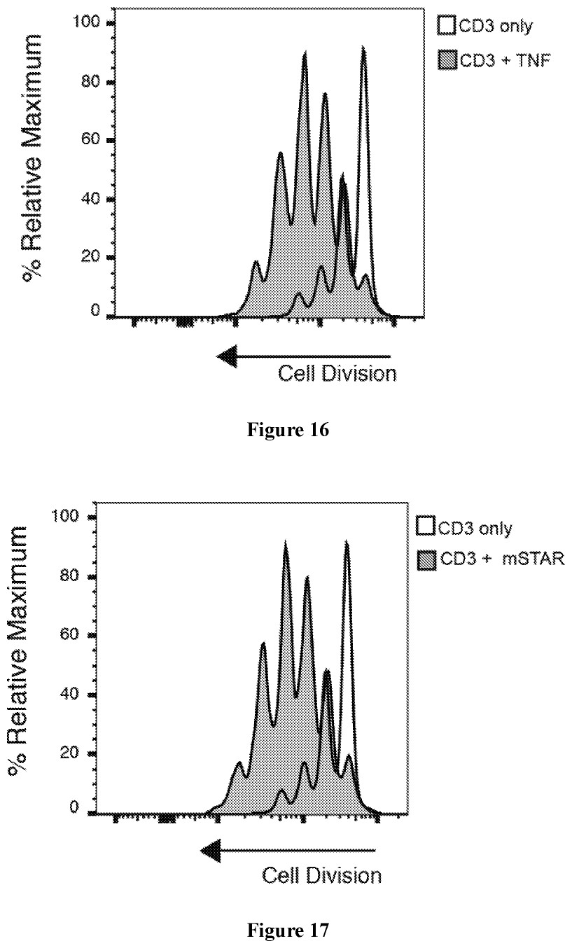

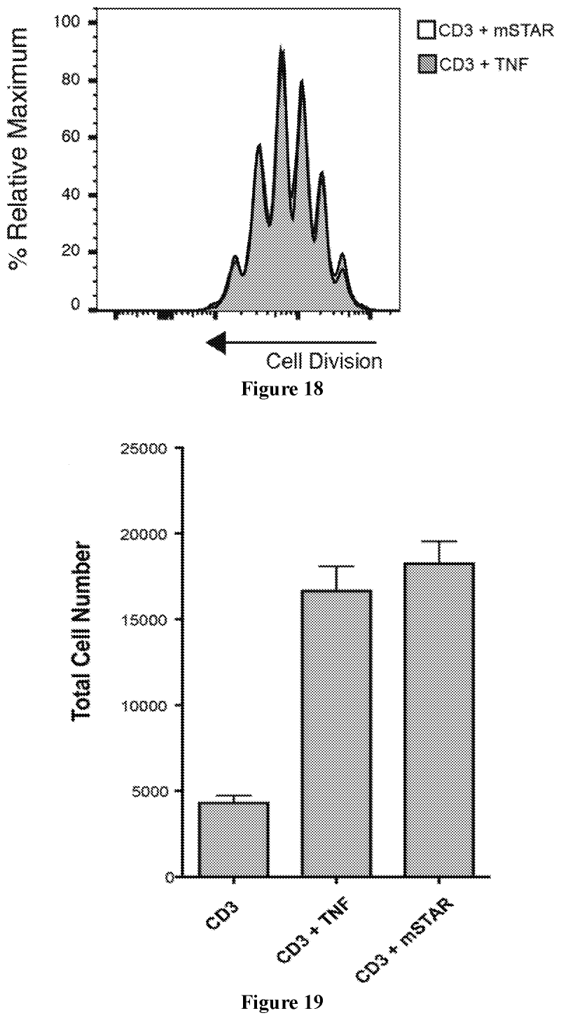

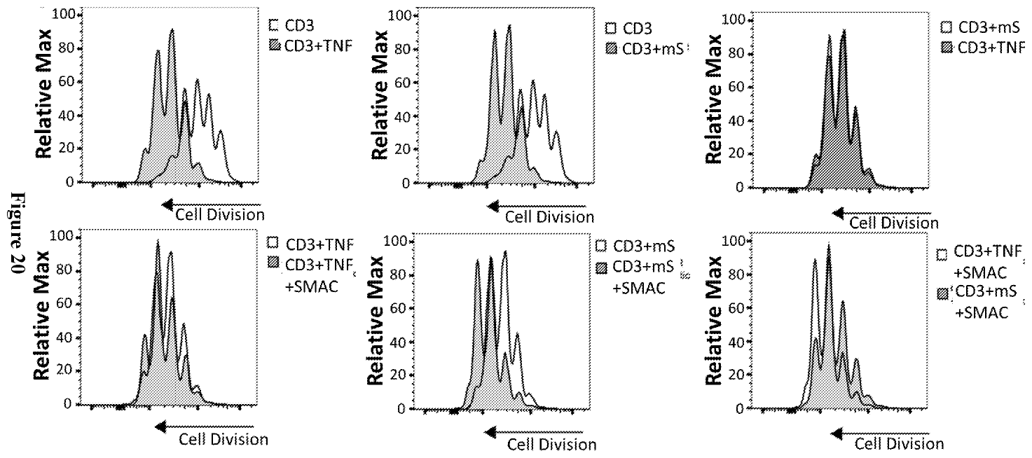

To determine if the co-stimulation effect of TNF on T cells could be mimicked using an agonist specific for TNFR2, the response of naïve mouse CD4 T cells activated via TCR in the presence of recombinant mouse TNF, or a TNFR2-specific agonist called mSTAR (Rauert et al., 2010; Chopra et al., 2016) was measured. Naïve CD4 T cells were CTV labelled and the response measured by flow cytometry after stimulation with anti-CD3 in the presence of TNF or mSTAR (FIGS. 16 to 19). Both TNF and mSTAR yielded a similar response in CD4 T cells as measured by dilution of cell tracking dyes (FIGS. 16 to 18) and total cell numbers (FIG. 19).

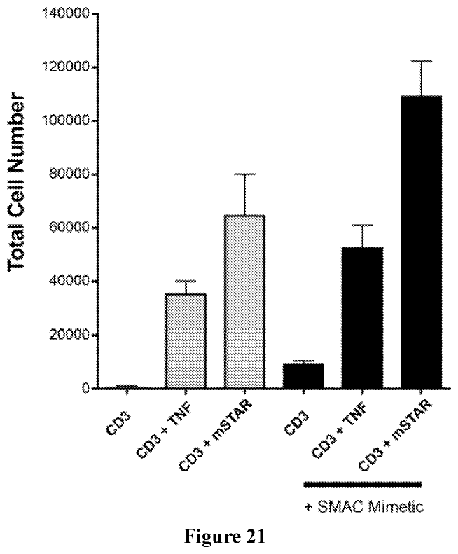

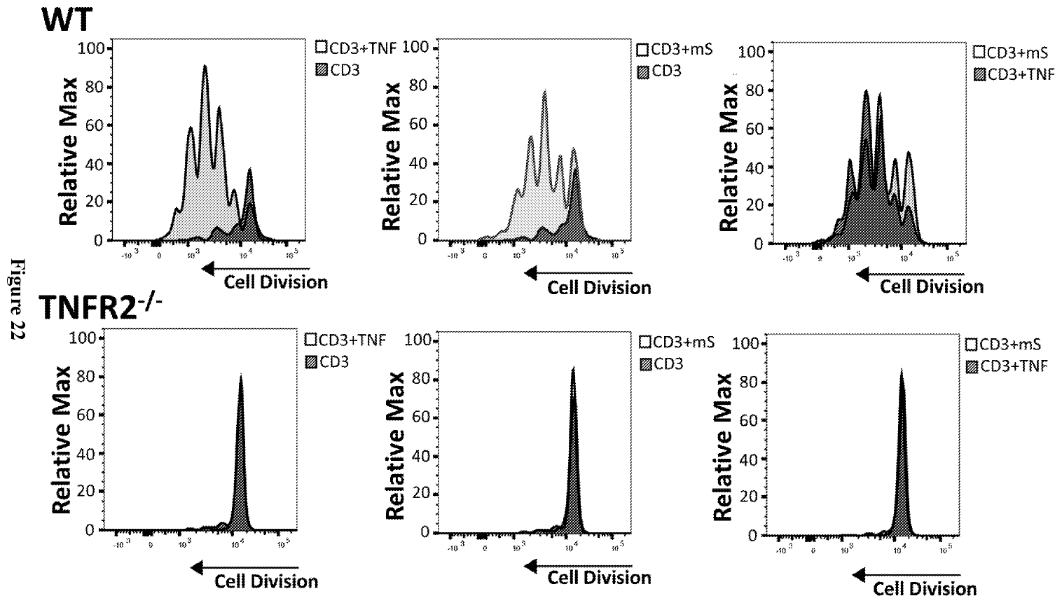

Similar results were also noted for CD8 T cells (FIG. 20 and FIG. 21) demonstrating that specifically targeting TNFR2 is a universal mechanism for enhancing (or in contrast with blocking reagents, dampening) the T cell response. Furthermore, SMAC mimetics could synergise to enhance the T cell response even in the presence of TNFR2-specific co-stimulation by mSTAR (FIG. 21).

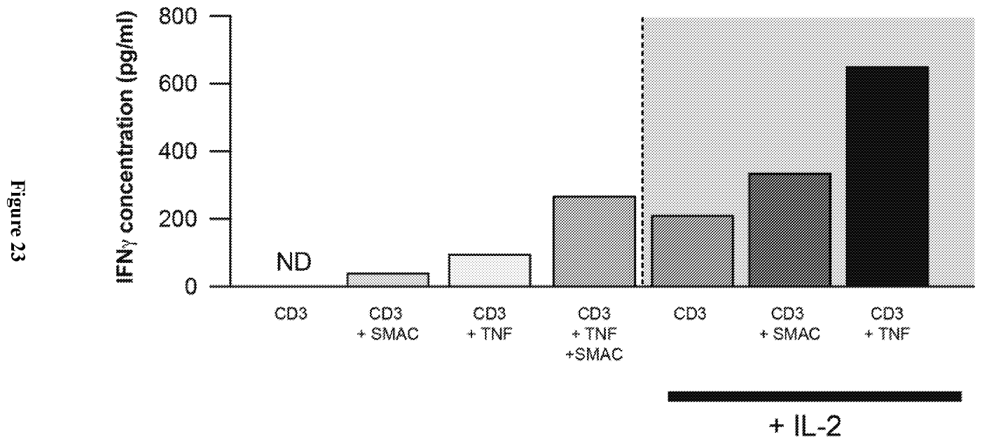

Furthermore, the TNFR2-specific agonist mSTAR did not induce any T cell co-stimulation in CD8 T cells deficient for TNFR2 demonstrating that enhancing the T cell response was specific to this T cell surface receptor (FIG. 22).

### Example 6

### Effect of TNFR2 Agonism on Cytokine Production

The effect of TNFR2 co-stimulation with TNF on the differentiation of TCR activated CD8 T cells was determined. Combined with enhancing T cell proliferation, TNF co-stimulation significantly increased the production of effector cytokines such as IFNγ (FIG. 23), even at 48 hours before T cell proliferation was observed by dilution of CTV. This effect was synergised by the addition of smac-mimetics or IL-2 (FIG. 23). Combined, these data suggest that targeting TNFR2 can be used either alone, or in combination with additional reagents to significantly modify immune responses to increase or reduce T cell activation as required in a disease specific manner.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from AU 2019901818, filed on 28 May 2019, the entire contents of which are incorporated herein by reference.

All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Abassi et al. (2003) J Biol Chem 278:35636-35643
Altman et al. (1996) Science 274:94-6
Amrani et al. (1996) Am J Respir Cell Mol Biol 15:55-63
Bercovici et al. (2000) Clin Diagn Lab Immunol 7:859-864
Caruso et al. (1997) J Quant Cell Sci 27:71-76
Chopra et al. (2016) J Exp Med 2016
Dal Porto et al. (1993) Proc Natl Acad Sci USA 90
Dong et al. (2016) PNAS 113:12304-12309
Fischer et al. (2011) PLoS ONE 6(11) e27621
Fischer et al. (2017) Scientific Reports 7:6607
Freedman et al. (1987) J Immunol 137:3260-3267
Freeman et al. (1989) J Immunol 143:2714-2722
Galloway et al. (1992) Eur J Immunol 22:3045-3048
Gilbert (2012) Immunology 135:19-26
Grell et al. (1995) Cell 83:793-802
Hamm-Alvarez et al. (2001) Am J Physiol Cell Physiol 280:C1657-C1668
He et al. (2016) PLos One 11: e0156311
June and Sadelain (2018) N Engl J Med 379:64-73
June et al. (2018) Science 359:1361-1365
Kureishi et al (2000) Nat Med 6:1004-1010
Laichalk et al. (1998) Infection & Immunity 66:2822-2826
Loetscher et al. (1993) J Biol Chem 268:26350
Miliotou and Papadopoulou (2018) Curr Pharm Biotechnol 19:5-18
Nie et al. (2018) Sci Signal 11:eaan0790
Okubo et al (2013) Sci Rep 3:3153
Okubo et al (2016) Immunology 5: e56
Pan et al (2002) Mol Cell Biol 22:7512-7523
Pardoll (2012) Nature Reviews Cancer 12:252-264
Plebanski et al. (2010) Expert Rev Vaccines 9:595-600
Rauert et al. (2010) J Biol Chem 285:7394-404
Slowik M R et al. (1993) Am J Pathol 143:1724-30
Smith et al. (1994) J Biol Chem 269:9898-9905
Tam et al. (2019) Sci. Transl. Med. 11, eaax0720
Tamagnone et al. (1994) Oncogene 9:3683-3688
Tartaglia et al. (1993) J Biol Chem 268:18542-18548
Tartaglia et al. (1993) J Immunol 151:4637-4641
Torrey et al. (2017) Sci Signal 10:eaaf8608
Van Ostade et al. (1991) EMBO J 10:827-36
Van Ostade et al. (1994) Eur J Biochem 220:771-9
Vanamee and Faustman (2017) Trends in Molecular Medicine 23:1037-1046
Wada et al. (2013) J Transl Med 11:89
Zhang et al. (2003) J Clin Invest 111:1933-1943

1. A method of increasing activation of effector T cells in a subject, the method comprising administering a TNFR2-specific agonist to the subject.

2. The method of claim 1, wherein the subject has a disease which would benefit from an increase in activation of effector T cells and the TNFR2-specific agonist is administered to treat the disease.

3. The method of claim 2, wherein the disease is a cancer, infection, or immunodeficiency.

4. The method of claim 3, wherein the cancer is lung cancer, breast cancer, colorectal cancer, prostate cancer, skin cancer (non-melanoma), melanoma, stomach cancer, pancreatic cancer, liver cancer, brain cancer, glioblastoma, neuroblastoma, blood cancer, acute myeloid leukaemia, acute lymphoblastic leukaemia, chronic lymphocytic leukemia, chronic myelogenous leukemia, a chronic myeloproliferative neoplasm, parathyroid cancer, renal cancer, retinoblastoma, rhabdomyosarcoma, savilary gland cancer, sarcoma, TNF-secreting T cell lymphoma, throat cancer, thymoma, thymic carcinoma, wilms tumour, hodgkin lymphoma, non-hodgkin lymphoma, merkel cell carcinoma, esophageal cancer, bladder cancer, bile duct cancer, bone cancer, Ewing sarcoma, osteosarcoma, malignant fibrous histiocytoma, multiple myeloma, or ovarian cancer.

5. The method of claim 3, wherein the infection is an acute or a chronic infection.

6. The method of claim 3 or claim 5, wherein the infection is a tuberculosis, influenza, mycobacterium uclercans, hepatitis, herpes simplex virus, ebola virus, human immunodeficiency virus, encephalitis, burkholderia pseudomallei, legionellosis, leishmaniasis, listeriosis, malaria, measles, meningococcal meningitis, pneumonia, salmonella, rubella, rabies, tetanus, typhoid, west nile virus, zika virus, anthrax, dengue fever, brucellosis, or campylobacter infection.

7. The method of any one of claims 1 to 6, wherein the TNFR2-specific agonist is or comprises a TNFR2-specific TNF mutein.

8. The method of claim 7, wherein the TNFR2-specific TNF mutein is mSTAR2, hSTAR2, sc-mTNF_{R2}, EHD2-sc-hTNF_{R2}, p53-sc-mTNF₂, GCN4-sc-mTNF_{R2}, TNC-scTNF_{R2}, or TNF (D143N/A145R).

9. The method of any one of claims 1 to 7, wherein the TNFR2-specific agonist is a polypeptide comprising a sequence which is at least 70% or at least 80% or at least 90% or at least 95% identical to SEQ ID NO:2 or SEQ ID NO:3 or a biologically active fragment thereof.

10. The method of claim 9, wherein the TNFR2-specific agonist is hSTAR2 or mSTAR2.

11. The method of any one of claims 1 to 7, wherein the TNFR2-specific agonist is a polypeptide comprising a sequence which is at least 70% or at least 80% or at least 90% or at least 95% identical to SEQ ID NO:4 or a biologically active fragment thereof.

12. The method of claim 11, wherein the TNFR2-specific agonist is EHD2-sc-hTNF_{R2}.

13. The method of any one of claims 1 to 7, wherein the TNFR2-specific agonist is a polypeptide comprising a sequence which is at least 70% or at least 80% or at least 90% or at least 95% identical to SEQ ID NO:5 or a biologically active fragment thereof.

14. The method of claim 13, wherein the TNFR2-specific agonist is TNC-scTNF R2.

15. The method of any one of claims 1 to 6, wherein the TNFR2-specific agonist is a polypeptide that comprises an antigen-binding domain which binds to TNFR2.

16. The method of claim 15, wherein the polypeptide is an antibody.

17. The method of any one of claims 1 to 16, wherein the TNFR2-specific agonist is administered to the subject in combination with another compound or cell.

18. The method of claim 17, wherein the other compound or cell is an immunotherapy.

19. The method of claim 18, wherein the immunotherapy is a T cell-based immunotherapy.

20. The method of claim 19, wherein the T cell-based immunotherapy is a CAR-T cell therapy.

21. The method of claim 17, wherein the other compound or cell is a vaccine and the TNFR2-specific agonist is administered as a vaccine adjuvant.

22. The method of claim 21, wherein the vaccine is a T cell vaccine.

23. The method of claim 17, wherein the other compound is a SMAC mimetic.

24. The method of claim 23, wherein the SMAC mimetic is birinapant, Debio 1143, CUDC-427, LCL161, AEG40826, ASTX-660, LBW-242, AZD5582, AEG40730, APG-1387, CompA, GDC-0145, GDC-0152, CS3, BV6, MV1, SM-164, AT406, ML101, or embelin.

25. The method of claim 24, wherein the SMAC mimetic is birinapant.

26. The method of claim 17, wherein the other compound is a T cell receptor agonist.

27. The method of claim 26, wherein the T cell receptor agonist is a Tribody.

28. The method of claim 27, wherein the tribody is Tb535.

29. The method of claim 17, wherein the other compound is a checkpoint inhibitor.

30. The method of claim 29, wherein the checkpoint inhibitor is a CTLA-4 antagonist, PD-1 antagonist, or PD-L1 antagonist.

31. The method of claim 29 or claim 30, wherein the checkpoint inhibitor is pembrolizumab, ipilimumab, nivolumab, or atezolizumab.

32. Use of a TNFR2-specific agonist in the manufacture of a medicament for increasing activation of effector T cells in a subject.

33. A TNFR2-specific agonist for use in increasing activation of effector T cells in a subject.

34. A method of inhibiting activation of effector T cells in a subject, the method comprising administering a TNFR2-specific antagonist to the subject.

35. The method of claim 34, wherein the subject has a disease mediated by over-activation of effector T cells, and the TNFR2-specific antagonist is administered to the subject to treat the disease.

36. The method of claim 35, wherein the disease is an autoimmune disease, inflammatory disease, or a non-TNF secreting T cell lymphoma.

37. The method of claim 35, wherein the disease is transplant rejection, graft versus host disease, inflammatory bowel disease, ulcerative colitis, lupus, polyarthritis, rheumatoid arthritis, reactive arthritis, osteomyelitis, toxic shock syndrome, psoriasis, Hidradenitis Suppurativa, ankylosing spondylitis, asthma, type 1 diabetes, type 2 diabetes, cardiovascular disease, or vasculitis.

38. The method of any one of claims 34 to 37, wherein the TNFR2-specific antagonist competitively inhibits TNFα binding to TNFR2.

39. The method of any one of claims 34 to 37, wherein the TNFR2-specific antagonist is a polypeptide that comprises an antigen-binding domain of an antibody.

40. The method of claim 39, wherein the polypeptide is an anti-TNFR2 antibody.

41. The method of claim 39 or claim 40, wherein the antigen binding domain comprises the CDRs of M861 or TR75-54.7.

42. The method of claim 41, wherein the antibody is M861 or TR75-54.7.

43. The method of any one of claims 34 to 42, wherein the TNFR2-specific antagonist is administered in combination with another compound or cell.

44. The method of claim 43, wherein the other compound is a TNFα antagonist.

45. The method of claim 44, wherein the TNFα antagonist is adalimumab, etanercept, golimumab, certolizumab, or infliximab.

46. The method of claim 43, wherein the other compound is a CD52 antagonist, CD20 antagonist, or IL-17A antagonist.

47. The method of claim 45, wherein the CD52 antagonist is alemtuzumab.

48. The method of claim 45, wherein the CD20 antagonist is rituximab.

49. The method of claim 45, wherein the IL-17A antagonist is ixekizumab.

50. Use of a TNFR2-specific antagonist in the manufacture of a medicament for inhibiting activation of effector T cells in a subject.

51. A TNFR2-specific antagonist for use in inhibiting activation of effector T cells in a subject.

52. The method of any one of claims 1 to 31 or 34 to 49, or the use of claim 32 or claim 50, or the TNFR2-specific agonist or antagonist of claim 33 or claim 51, wherein the subject is a human.

53. An in vitro method of expanding a population of effector T cells, the method comprising contacting the effector T cells with a TNFR2-specific agonist.

54. A composition comprising at least two agents for increasing activation of effector T cells, wherein one of the agents is a TNFR2-specific agonist.

55. A composition comprising at least two agents for treating cancer, infection or an immunodeficiency, wherein one of the agents is a TNFR2-specific agonist.

56. A composition comprising at least two agents for inhibiting activation of T cells in a subject, wherein one of the agents is a TNFR2-specific antagonist.

57. A composition comprising at least two agents for treating an autoimmune disease, inflammatory disease or non-TNF secreting T cell lymphoma, wherein one of the agents is a TNFR2-specific antagonist.

58. A kit comprising at least two agents for increasing activation of effector T cells, wherein one of the agents is a TNFR2-specific agonist.

59. A kit comprising at least two agents for treating cancer, infection or an immunodeficiency, wherein one of the agents is a TNFR2-specific agonist.

60. A kit comprising at least two agents for inhibiting activation of T cells in a subject, wherein one of the agents is a TNFR2-specific antagonist.

61. A kit comprising at least two agents for treating an autoimmune disease, inflammatory disease or non-TNF secreting T cell lymphoma, wherein one of the agents is a TNFR2-specific antagonist.

62. The method of any one of claim 1 to 31, 52 or 53, the use of claim 32, the TNFR2-specific agonist of claim 33, the composition of claim 54 or claim 55, or the kit of claim 58 or claim 59, wherein the TNFR2-specific agonist
a) binds to TNFR2 with an affinity which is at least 1.2-fold, at least 1.5-fold, at least 2-fold, at least 5-fold, or at least 10-fold stronger than its affinity for binding to TNFR1, and/or
b) activates TNFR2 signaling at a level which is at least 1.2-fold, at least 1.5-fold, at least 2-fold, at least 5-fold, or at least 10-fold higher than it activates TNFR1 signaling.

63. The method of any one of claim 34 to 49 or 52, the use of claim 50, the TNFR2-specific antagonist of claim 51, the composition of claim 56 or claim 57, or the kit of claim 60 or claim 61, wherein the TNFR2-specific antagonist
a) binds to TNFR2 with an affinity which is at least 1.2-fold, at least 1.5-fold, at least 2-fold, at least 5-fold, or at least 10-fold stronger than its affinity for binding to TNFR1, and/or
b) inhibits TNFR2 signaling at a level of inhibition which is at least at least 1.2-fold, 1.5-fold, at least 2-fold, at least 5-fold, or at least 10-fold higher than it inhibits TNFR1 signaling.


SEQUENCE LISTING (14 sequences)

SEQ ID NO: 1 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 2 - protein, 515 aa, Artificial Sequence
  Synthetic protein
ACGCAAAPDIKDLLSRLEELEGLVSSLREQGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 3 - protein, 513 aa, Artificial Sequence
  Synthetic protein
ACGCAAAPDIKDLLSRLEELEGLVSSLREQGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIALGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIALGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIAL

SEQ ID NO: 4 - protein, 658 aa, Artificial Sequence
  Synthetic protein
METDTLLLWVLLLWVPGSTGDAAQPAGGGAAAHHHHHHGGTGGGGSGGKLGGSGGDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNGGGSGGGTGSEFLASSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 5 - protein, 516 aa, Artificial Sequence
  Synthetic sequence
ACGCAAAPDVKELLSRLEELENLVSSLREQGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 6 - protein, 156 aa, Artificial Sequence
  Synthetic sequence
LRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIAL

SEQ ID NO: 7 - protein, 679 aa, Artificial Sequence
  Synthetic protein
KKPLDGEYFTLQIRGRERFEMFRELNEALELKDAQAGKEPLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIALGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIALGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIALGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIAL

SEQ ID NO: 8 - protein, 833 aa, Artificial Sequence
  Synthetic sequence
RMKQLEDKVEELLSKNYHLENEVARLKKLVGERLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIALGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIALGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIALGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIALGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIAL

SEQ ID NO: 9 - protein, 157 aa, Artificial Sequence
  Synthetic sequence
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 10 - protein, 652 aa, Artificial Sequence
  Synthetic protein
METDTLLLWVLLLWVPGSTGDAAQPAGGGAAAHHHHHHGGTGGGGSGGKLGGSGGAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDGGGSGGGTGSEFLASSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 11 - protein, 647 aa, Artificial Sequence
  Synthetic sequence
METDTLLLWVLLLWVPGSTGDAAQPAGGGAAAHHHHHHGGTGGGGSGGKLGGSGGDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNGGGSGGGTGSEFLASSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 12 - protein, 660 aa, Artificial Sequence
  Synthetic sequence
METDTLLLWVLLLWVPGSTGDAAQPAGGGAAAHHHHHHGGTGGGGSGGKLGGSGGDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNGGGSGGGSGGGSGGGSGGGSGGSEFLASSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIAL

SEQ ID NO: 13 - protein, 157 aa, Artificial Sequence; identical to SEQ ID NO: 9
  Synthetic protein

SEQ ID NO: 14 - protein, 655 aa, Artificial Sequence
  Synthetic protein
METDTLLLWVLLLWVPGSTGDAAQPAGGGAAAHHHHHHGGTGGGGSGGKLGGSGGDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNGGGSGGGTGSEFLASSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIALGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIALGGGGSLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIAL